SEQUENCE LISTING (8 sequences)

SEQ ID NO: 1 - protein, 461 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCGLUSRWGGDGFYAMDYWGQGTLVTVSSASTKGPSVFPLAPSSKSTSGGTAALGCGLULVKDYFPEPVTVSWNSGALTSGVHTFPAVLQSSGLYSLSSVVTVPSSSLGTQTYICNVNHKPSNTKVDKKVEPKSCDKTHTCGLUPPCGLUPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 217 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGLU

SEQ ID NO: 3 - protein, 246 aa, synthetic construct
ATGGGCAGCAGCCATCATCATCATCATCACAGCAGCGGCGAAAACCTGTATTTTCAGGGCCATATGGTTGATAACAAATTTAACAAAGAAATGCGCAACGCATATTGGGAAATTGCACTGCTGCCGAATCTGAATAATCAGCAGAAACGTGCGTTTATTCGTAGCCTGTATGATGATCCGAGTCAGAGCGCAAACCTGCTGGCAGAAGCAAAAAAACTGAATGATGCACAGGCACCGAAATGCTAG

SEQ ID NO: 4 - protein, 81 aa, synthetic construct
MGSSHHHHHHSSGENLYFQGHMVDNKFNKEMRNAYWEIALLPNLNNQQKRAFIRSLYDDPSQSANLLAEAKKLNDAQAPKC

SEQ ID NO: 5 - protein, 84 aa, synthetic construct
MGSSHHHHHHSSGENLYFQGHMVDNKFNKEMRNAYWEIALLPNLNNQQKRAFIRSLYDDPSQSANLLAEAKKLNDAQAPKCGLU

SEQ ID NO: 6 - protein, 10 aa, synthetic construct
CCRGDKGPDC

SEQ ID NO: 7 - protein, 13 aa, synthetic construct
CXYLCRGDKGPDC

SEQ ID NO: 8 - protein, 19 aa, synthetic construct
CXYLCGLURGDKGPDCGLU

(19) United States
(12) Patent Application Publication
(10) Pub. No.: US 2025/0042936 A1
(43) Pub. Date: Feb. 6, 2025

(54) METHOD FOR GLYCOSYLATION MODIFICATION OF PROTEINS AND/OR POLYPEPTIDES

(71) Applicant: SICHUAN UNIVERSITY, Chengdu, Sichuan (CN)

(72) Inventors: Dawen NIU, Chengdu (CN); Xia ZHANG, Chengdu (CN); Chen ZHANG, Chengdu (CN); Hao ZUO, Chengdu (CN); Hongxin ZENG, Chengdu (CN); Yanjing LI, Chengdu (CN); Jiaxi WANG, Chengdu (CN); Haiyan REN, Chengdu (CN)

(21) Appl. No.: 18/915,740

(22) Filed: Oct. 15, 2024

Related U.S. Application Data
(63) Continuation-in-part of application No. PCT/CN2022/118051, filed on Sep. 9, 2022.

(51) Int. Cl. C07K 1/107

(52) U.S. Cl. CPC C07K 1/1077

(57) ABSTRACT

A method for the glycosylation modification of proteins and/or polypeptides uses glycosyl sulfinate as a raw material. The method for glycosylation and modification is applicable not only to proteins and/or peptides containing disulfide bonds, but also to proteins and/or peptides containing sulfhydryl groups. The method is even applicable to proteins and/or peptides containing both disulfide bonds and sulfhydryl groups.

### INCORPORATION OF SEQUENCE LISTING

This application contains a sequence listing submitted in Computer Readable Form (CRF). The CRF file contains the sequence listing entitled “PA288.0148-SequenceListing.xml”, which was created on Oct. 11, 2024, and is 8,600 bytes in size. The information in the sequence listing is incorporated herein by reference in its entirety.

### FIELD OF THE INVENTION

The present invention relates to the field of medicinal chemistry, and specifically to a method for glycosylation modification of proteins and/or polypeptides.

### BACKGROUND OF THE INVENTION

Glycosylation is a major post-translational modification of proteins. The cell surface of all living organisms is coated by many different types of sugar chains, and various kinds of glycosylation also occur within the cells. Glycoproteins are basic substances in many biological processes, including cell growth, cell to cell adhesion, immune response, fertilization, clot degradation, viral proliferation, parasitic infections, and inflammatory responses. With the completion of the Human Genome Project and the continuous development of proteomics technology, the study of glycosylated proteomics has received increasing attention.

Currently, the researches on protein glycosylation engineering are mostly focused on medicaments and food. It has been found that protein glycosylation can increase the half-life and targeting of recombinant protein drugs; glycosylation of fish protein, ovalbumin, β-lactoglobulin, bovine serum albumin, gluten and other proteins reveals that the newly synthesized glycoproteins exhibit varying degrees of improvement in functional properties such as emulsibility, solubility, thermal stability, antibacterial and antioxidant properties, etc; the research results on the nutrition and toxicology of modified glycoproteins indicate that glycosylation reaction can inhibit the allergic reaction characteristics of β-lactoglobulin to some extent. For essential amino acids in proteins, except for minor loss of lysine, other amino acids are almost not affected. Therefore, it is of great significance to study the methods for glycosylation modification of proteins.

At present, the methods for protein glycosylation mainly include dry-heat method and wet-heat method. Among them, the dry-heat method for glycosylation is the first method used for protein glycosylation, and is also the most important method for glycosylation treatment of proteins. Usually, proteins and polysaccharides are mixed in a certain proportion in an aqueous solution, dried to obtain their mixed powder, and then glycosylation reaction is induced under conditions of certain temperature, humidity, and time. After completion of the reaction, the mixture is quickly cooled to terminate the glycosylation reaction. Dry-heat method for glycosylation has some advantages, such as simple operation, easy control of reaction conditions, no addition of other reagents, and high grafting degree of reaction products. However, the reaction time is relatively long, usually ranging from a few days to several weeks. Wet-heat method for glycosylation of proteins is a method for glycosylation modification of proteins based on the heat treatment of proteins and sugars in liquid phase, which is commonly used for grafting reactions between proteins and small molecule sugars. The wet-heat method for glycosylation has the advantages including short reaction time and rapid reaction. However, on the one hand, due to the reversible nature of the primary reaction in the Maillard reaction, water, as the reaction product of the primary reaction, is abundant in the system, which inhibits the progress of glycosylation reaction; on the other hand, proteins undergoing high-temperature treatment in water are prone to denaturation and aggregation, accelerating the reaction towards advanced stages and even generating toxic and harmful substances such as acrylamide and 4-methylimidazole. Therefore, the wet-heat method for glycosylation has some problems, including incomplete reaction, low grafting degree, complex products, and difficult reaction control.

It is of great significance to develop a method for glycosylation modification of proteins or polypeptides with the advantages of easily available reaction materials, mild reaction conditions, short reaction time, and controllable reaction process.

### CONTENT OF THE INVENTION

The object of the present invention is to provide a new method for glycosylation modification of proteins or polypeptides with the advantages of easily available reaction materials, mild reaction conditions, short reaction time, and controllable reaction process.

The present invention provides a method for glycosylation modification of proteins and/or polypeptides, characterized in that the method comprises the following steps:
the starting materials including glycosyl sulfinates, proteins and/or peptides, and oxidants are allowed to react in a solvent, to obtain glycosylation-modified proteins and/or peptides;
wherein, the proteins and/or polypeptides contain sulfhydryl and/or disulfide bonds;
the structure of the glycosyl sulfinate is as represented by formula W:

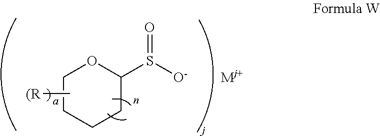

wherein, n is selected from 0 or 1;
a is selected from 3 or 4;
R is each independently selected from L₁Rₓ; or two adjacent R are linked to form a ring, while the other R is each independently selected from L₁Rₓ. The ring is either unsubstituted or substituted with one or more L₁Rₓ;
L₁ is selected from absence or C₁₋₂ alkylene, and Rₓ is selected from the group consisting of H, OH, C₁₋₆ alkyl, OAc (acetyloxy), OBn (benzyloxy), OR₈, NR₉R₁₀,

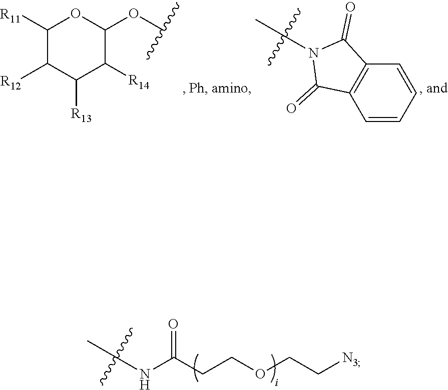

i is an integer selected from 0 to 6;
R₁₁, R₁₂, R₁₃, and R₁₄ are each independently selected from L₂R_{y}; L₂ is selected from absence or C₁₋₂ alkylene, and R_{y} is selected from the group consisting of H, OH, C₁₋₆ alkyl, OAc, OBn, OR₈, and NR₉R₁₀;
R₈ is selected from C₁₋₆ alkyl;
R₉ is selected from the group consisting of H, C₁₋₆ alkyl, Ac (acetyl), and Bn (benzyl);
R₁₀ is selected from the group consisting of H, C₁₋₆ alkyl, Ac, and Bn;
j is selected from 1, 2, or 3;
M^{j+} is a j-valent cation.

Further, the oxidant is selected from one or more of hydrogen peroxide, tert-butyl hydroperoxide, potassium persulfate, oxygen, and tert-butyl peroxide;
and/or, the solvent is an aqueous solution, and preferably water or buffer;
and/or, the temperature of the reaction is room temperature;
and/or, the reaction is carried out under inert gas atmosphere.

Further, the protein and/or polypeptide contains sulfhydryl, and the number of amino acid residues in the protein and/or polypeptide is 70-1000;
the method is method 1 or method 2;
method 1 comprises the following steps: first, the raw materials proteins and/or polypeptides containing sulfhydryl and compound A are allowed to react as the first step in a solvent, and then glycosyl sulfinates and oxidants are added for carrying out the second step reaction, to obtain glycosylation-modified proteins and/or polypeptides; compound A is

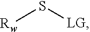

wherein LG is a leaving group, R_{w} is alkyl, aryl, or heteroaryl; alternatively, R_{w} and LG are linked to form a ring; preferably, compound A is

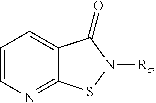

wherein R_{z} is C₁₋₈ alkyl, and preferably C₁₋₃ alkyl;
method 2 comprises the following steps: starting materials proteins and/or polypeptides containing sulfhydryl, glycosyl sulfinates, and oxidants are allowed to react in a solvent, to obtain glycosylation-modified proteins and/or polypeptides.
further, the molar ratio of the protein and/or polypeptide containing sulfhydryl, compound A, glycosyl sulfinate, and oxidant is 1:(10-300):(20-600):(20-600), and preferably 1:200:400:400; and/or, the ratio of the protein and/or polypeptide containing sulfhydryl to the solvent is (0.05-0.5) μmol:1 mL, and preferably 0.1 μmol:1 mL; and/or, the time for the first step reaction is 1-20 minutes, and preferably 10 minutes; the time for the second step reaction is 0.2-1.5 hours, and preferably 1 hour;

In method 2, the molar ratio of the protein and/or polypeptide containing sulfhydryl, glycosyl sulfinate, and oxidant is 1:(20-600):(20-600), and preferably 1:400:400; and/or, the ratio of the protein and/or peptide containing sulfhydryl to the solvent is (0.05-0.5) μmol:1 mL, and preferably 0.1 μmol:1 mL; and/or, the reaction time is 0.2-1.5 hours, and preferably 1 hour.

Further, the protein and/or polypeptide containing sulfhydryl is selected from the group consisting of thiol-containing Affibody, Mucin 1 protein, GTPase, sulfhydryl-containing non-structural protein, and sulfhydryl-containing amyloid protein;
preferably, the amino acid sequence of the sulfhydryl-containing Affibody is as set forth in SEQ ID NO.4.

Further, the protein and/or polypeptide contains sulfhydryl, and the number of amino acid residues in the protein and/or polypeptide is 1-100;
the method is method 3 or method 4;
method 3 comprises the following steps: first, the raw materials proteins and/or polypeptides containing sulfhydryl and compound A are allowed to react in a solvent as the first step, and then glycosyl sulfinates and oxidants are added for carrying out the second step reaction, to obtain glycosylation-modified proteins and/or polypeptides; compound A is

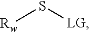

wherein LG is a leaving group, R_{w} is alkyl, aryl, or heteroaryl; alternatively, R_{w} and LG are linked to form a ring; preferably, compound A is

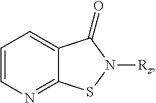

wherein R_{z} is C₁₋₈ alkyl, and preferably C₁₋₃ alkyl;
method 4 comprises the following steps: starting materials proteins and/or polypeptides containing sulfhydryl, glycosyl sulfinates, and oxidants are allowed to react in a solvent, to obtain glycosylation-modified proteins and/or polypeptides.

Further, in method 3, the molar ratio of the protein and/or polypeptide containing sulfhydryl, compound A, glycosyl sulfinate, and oxidant is 1:(1-3):(3-10):(3-10), and preferably 1:3:6:6; and/or, the ratio of the protein and/or polypeptide containing sulfhydryl to the solvent is (0.01-0.2) μmol:1 mL, and preferably 0.01 μmol:1 mL; and/or, the time for the first step reaction is 1-20 minutes, and preferably 10 minutes; the time for the second step reaction is 0.2-1.5 hours, and preferably 1 hour;
in method 4, the molar ratio of the protein and/or polypeptide containing sulfhydryl, glycosyl sulfinate, and oxidant is 1:(3-10):(3-10), and preferably 1:6:6; and/or, the ratio of the protein and/or polypeptide containing sulfhydryl to the solvent is (0.01-0.2) μmol:1 mL, and preferably 0.01 μmol:1 mL; and/or, the reaction time is 0.2-1.5 hours, and preferably 1 hour.

Further, the protein and/or polypeptide containing sulfhydryl is selected from the group consisting of αVβ integrin-binding peptide, cell-penetrating peptide-R8, and reduced glutathione

Further, the protein and/or polypeptide contains disulfide bonds, and the number of amino acid residues in the protein and/or polypeptide is 2-2000;
the method comprises the following steps: glycosyl sulfinates, proteins and/or polypeptides containing disulfide bonds, and oxidants are allowed to react in a solvent, to obtain glycosylation-modified proteins and/or polypeptides.

Further, the molar ratio of the protein and/or polypeptide containing disulfide bonds, glycosyl sulfinate, and oxidant is 1:(60-600):(20-600), and preferably 1:400:400;
and/or, the ratio of the protein and/or polypeptide containing disulfide bonds to the solvent is (0.05-0.5) μmol: 1 mL, and preferably 0.1 μmol: 1 mL;
and/or, the reaction time is 0.2-1.5 hours, and preferably 0.5-1 hour.

Further, the protein and/or polypeptide containing disulfide bonds is selected from the group consisting of Herceptin, Inotuzumab ozogamicin, TGuard protein, Brentuximab vedotin, Mirvetuximab soravtansine, Upifitamab rilsodotin, Enfortumab vedotin, Certolizumab Gleevec, Telisotuzumab vedotin, Tusamitamab ravtansine, Ravtansine for treating thyroid adenomas, Recaticimab, amyloid P/A4 protein, Jag1 protein, lysozyme, iRGD peptide, and insulin.

Further, the protein and/or polypeptide contains sulfhydryl and disulfide bonds;
the method is method 5 or method 6;
method 5 includes the following steps: first, proteins and/or polypeptides containing sulfhydryls and disulfide bonds, and compound A are used as raw materials for the first step and allowed to react in a solvent; and then glycosyl sulfinate a and oxidant are added to carry out the second step reaction, so as to obtain the proteins and/or polypeptides modified with glycosyl sulfinate a; and then glycosyl sulfinate b and oxidant are added to carry out the third step reaction, so as to obtain the proteins and/or polypeptides modified with glycosyl sulfinates a and b; compound A is

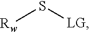

wherein LG is a leaving group, R_{w} is alkyl, aryl, or heteroaryl; alternatively, R_{w} and LG are linked to form a ring; preferably, compound A is

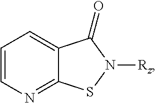

wherein R_{z} is C₁₋₈ alkyl, and preferably C₁₋₃ alkyl;
method 6 includes the following steps: firstly, using proteins and/or polypeptides containing sulfhydryls and disulfide bonds, glycosyl sulfinate a, and oxidants as raw materials, the first step reaction is carried out in a solvent to obtain proteins and/or polypeptides modified with glycosyl sulfinate a; then, glycosyl sulfinate b and oxidants are added to carry out the reaction in step (2), so as to obtain proteins and/or polypeptides modified with glycosyl sulfinates a and b;
glycosyl sulfinate a is the above glycosyl sulfinate, while glycosyl sulfinate b is the above glycosyl sulfinate, and glycosyl sulfinate a and glycosyl sulfinate b are the same or different.

Further, in method 5, the molar ratio of the protein and/or polypeptide containing sulfhydryls and disulfide bonds, compound A, glycosyl sulfinate a, glycosyl sulfinate b, the oxidant used in the second step reaction, and the oxidant used in the third step reaction is 1:(1-3):(1-3):(1-3):(1-3):(1-3), and preferably 1:1.2:1.2:2:1.6:2; and/or, the ratio of the protein and/or polypeptide containing sulfhydryls and disulfide bonds to the solvent is (0.001-0.01) mmol:1 mL, and preferably 0.005 mmol:1 mL; and/or, the time for the first step reaction is 5-20 minutes, and preferably 10 minutes; the time for the second step reaction is 0.2-1.5 hours, and preferably 1 hour; the time for the third step reaction is 0.2-1.5 hours, and preferably 1 hour;
in method 6, the molar ratio of the protein and/or polypeptide containing sulfhydryls and disulfide bonds, glycosyl sulfinate a, glycosyl sulfinate b, the oxidant used in the first step reaction, and the oxidant used in the second step reaction is 1:(1-3):(1-3):(1-3):(1-3), and preferably 1:1.2:2:1.6:2; and/or, the ratio of the protein and/or polypeptide containing sulfhydryls and disulfide bonds to the solvent is (0.001-0.01) mmol:1 mL, and preferably 0.005 mmol:1 mL; and/or, the time for the first step reaction is 0.2-1.5 hours, and preferably 1 hour; the time for the second step reaction is 0.2-1.5 hours, and preferably 1 hour;

Further, the protein and/or polypeptide containing sulfhydryl and disulfide bonds is selected from the polypeptides having the amino acid sequence of CCRGDKGPDC, the polypeptides having the amino acid sequence of SKDACIRTCVMCDEQ, and Sublantin antimicrobial peptides.

Further, the structure of the glycosyl sulfinate is as represented by formula I:

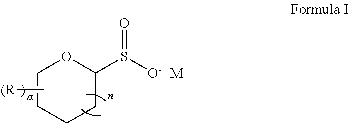

wherein, n is selected from 0 or 1;
a is selected from 3 or 4;
R is each independently selected from L₁Rₓ; alternatively, two adjacent R are linked to form a ring, while the other R is each independently selected from L₁Rₓ. The ring is either unsubstituted or substituted with one or more L₁Rₓ;
L₁ is selected from absence or C₁₋₂ alkylene, and Rₓ is selected from the group consisting of H, OH, C₁₋₆ alkyl, OAc, OBn, OR₈, NR₉R₁₀,

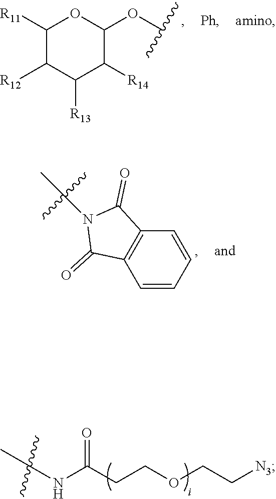

i is an integer selected from 0 to 6;
R₁₁, R₁₂, R₁₃, and R₁₄ are each independently selected from L₂R_{y}; L₂ is selected from absence or C₁₋₂ alkylene, and R_{y} is selected from the group consisting of H, OH, C₁₋₆ alkyl, OAc, OBn, OR₈, and NR₉R₁₀;
R₈ is selected from C₁₋₆ alkyl;
R₉ is selected from the group consisting of H, C₁₋₆ alkyl, Ac, and Bn;
R₁₀ is selected from the group consisting of H, C₁₋₆ alkyl, Ac, and Bn;
M⁺ is a monovalent cation.

Further, the structure of the glycosyl sulfinate is as represented by formula II:

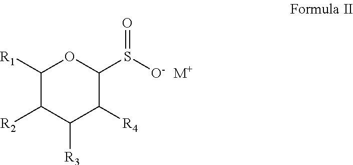

wherein, R₁, R₂, R₃, and R₄ are each independently selected from L₁Rₓ; alternatively, for R₁, R₂, R₃, and R₄, two adjacent groups are linked to form a ring, and the remaining two groups are each independently selected from L₁Rₓ. The ring is a 5-6-membered ring which is unsubstituted or substituted with one or more L₁Rₓ;
L₁ is selected from absence or methylene, and Rₓ is selected from the group consisting of H, OH, C₁₋₅ alkyl, OAc, OBn, OR₈, NR₉R₁₀,

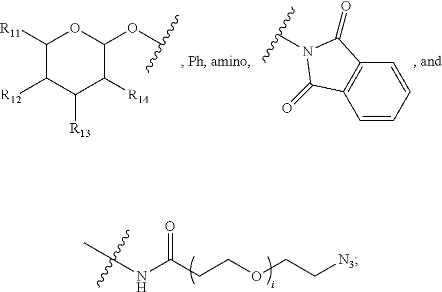

i is an integer selected from 0 to 4;
R₁₁, R₁₂, R₁₃, and R₁₄ are each independently selected from L₂R_{y}; L₂ is selected from absence or methylene, and R_{y} is selected from the group consisting of H, OH, C₁₋₅ alkyl, OAc, OBn, OR₈, and NR₉R₁₀;
R₈ is selected from C₁₋₅ alkyl;
R₉ is selected from the group consisting of H, C₁₋₅ alkyl, Ac, and Bn;
R₁₀ is selected from the group consisting of H, C₁₋₅ alkyl, Ac, and Bn;
M⁺ is a monovalent cation.

Further, the structure of the glycosyl sulfinate is as represented by formula III:

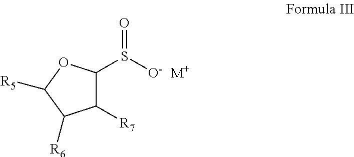

wherein, R₅, R₆, and R₇ are each independently selected from L₁Rₓ; alternatively, for R₅, R₆, and R₇, two adjacent groups are linked to form a ring, and the remaining group is L₁Rₓ. The ring is a 5-6-membered ring which is unsubstituted or substituted with one or more L₁Rₓ;
L₁ is selected from absence or methylene, and Rₓ is selected from the group consisting of H, OH, C₁₋₅ alkyl, OAc, OBn, OR₈, NR₉R₁₀,

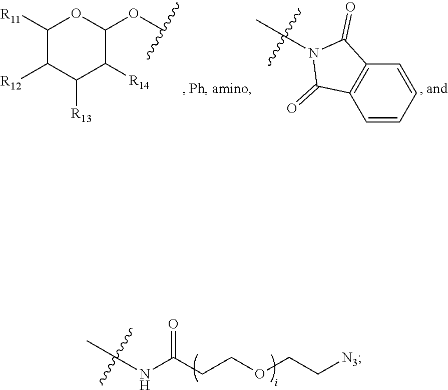

i is an integer selected from 0 to 4;
R₁₁, R₁₂, R₁₃, and R₁₄ are each independently selected from L₂R_{y}; L₂ is selected from absence or methylene, and R_{y} is selected from the group consisting of H, OH, C₁₋₅ alkyl, OAc, OBn, OR₈, and NR₉R₁₀;
R₈ is selected from C₁₋₅ alkyl;
R₉ is selected from the group consisting of H, C₁₋₅ alkyl, Ac, and Bn;
R₁₀ is selected from the group consisting of H, C₁₋₅ alkyl, Ac, and Bn;
M⁺ is a monovalent cation.

Further, the 5-6-membered ring is a 5-6-membered saturated oxygen-containing heterocycle;
R₈ is selected from C₁₋₃ alkyl;
R₉ is selected from the group consisting of H, C₁₋₃ alkyl, Ac, and Bn;
R₁₀ is selected from the group consisting of H, C₁₋₃ alkyl, Ac, and Bn;
M⁺ is selected from the group consisting of Na⁺, K⁺, and Li⁺.

Further, the structure of the glycosyl sulfinate is selected from the group consisting of:

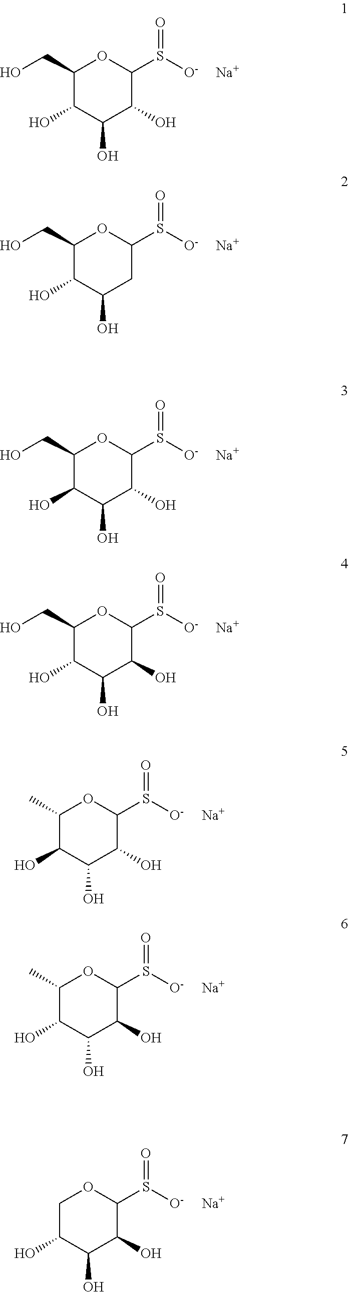

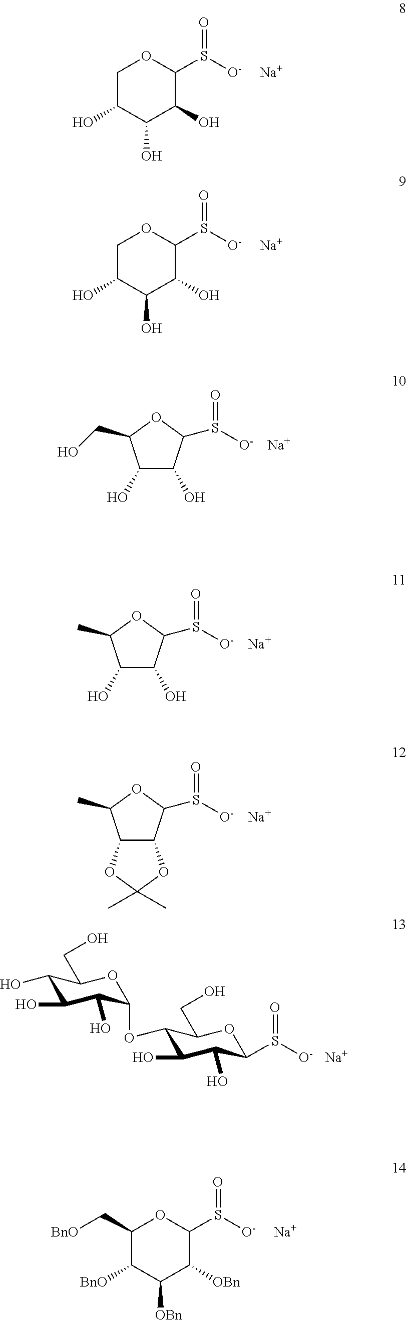

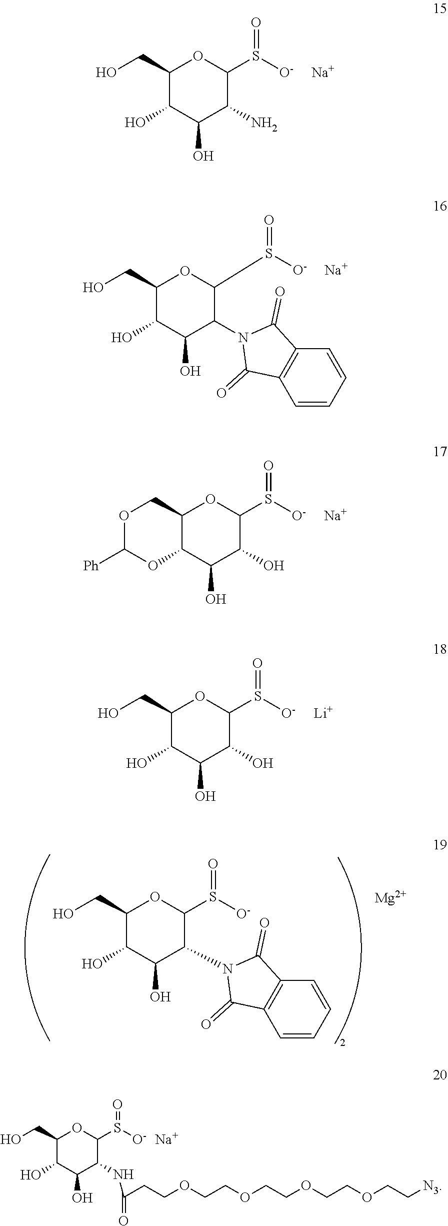

The present invention also provides the glycosylation-modified proteins and/or polypeptides prepared by the methods mentioned above.

For the definition of terms used in the present invention: unless defined otherwise, the initial definition provided for the group or term herein applies to the group or term of the whole specification; for the terms that are not specifically defined herein, based on the disclosed content and context, they should have the same meaning as commonly understood by one of ordinary skill in the art to which the disclosure belongs.

Proteins are bio-macromolecules composed of one or more long-chains of α-amino acid residues. α-amino acid molecules are in a linear arrangement, and the carboxyl and amino groups of adjacent α-amino acid residues are linked by a peptide bond, and finally folded to form a functional three-dimensional structure. The α-amino acid sequence of proteins is encoded by corresponding genes. In addition to the 20 “standard” amino acids encoded by the genetic code, certain α-amino acid residues in proteins can also undergo chemical structural changes by altering the order of atoms, thereby activating or regulating the protein. Multiple proteins and minerals can form stable protein complexes, often by binding together. Such large molecular structures can mechanically perform a specific function.

Polypeptides refer to short chains of amino acids linked by peptide bonds. Polypeptides belong to a broad chemical category of biopolymers and oligomers. Polypeptides are naturally occurring small biomolecules, that are substances of larger than amino acids but smaller than proteins. Amino acids have the smallest molecular weight, proteins have the largest molecular weight, and peptides are short chains composed of amino acids which are linked by peptide (amide) bonds. When the carboxyl group of one amino acid reacts with the amino group of another amino acid, the covalent chemical bond is formed. Dipeptides are protein fragments composed of two amino acids. Two or more amino acids undergo dehydration and condensation to form several peptide bonds and consist of a polypeptide. Multiple peptides undergo multi-level folding to constitute a protein molecule.

Antibodies refer to proteins that have protective effects produced by the body due to antigen stimulation.

Antibody refers to an antibody in which the terminal amino acid has been artificially modified.

Mucin-1 protein refers to mucoprotein-1.

GTPase refers to guanosine triphosphatase.

Nonstructural proteins refer to the proteins encoded by the viral genome that have certain functions during viral replication or gene expression regulation, but ultimately do not bind to form mature viruses and are not part of the viral structure.

Amyloid proteins refer to the glycoproteins derived from immunoglobulins that accumulate in tissues during amyloid degeneration.

Amino acid residues refer to the remaining portion of amino acids linked by peptide bonds after dehydration. Amino acids that consist of proteins or polypeptides lose one molecule of water when they bind to each other due to the participation of some of their functional groups in the formation of peptide bonds. Therefore, the amino acid units in polypeptides are called amino acid residues.

Sulfhydryl group refers to —SH, which can be a sulfhydryl derived from cysteine; disulfide bond refers to —S—S—, which can be a disulfide bond formed by covalently linking two cysteine residues.

For the groups of the present invention, Ac represents acetyl; Bn represents benzyl; Ph represents phenyl; Me represents methyl.

The present invention provides a new method for glycosylation modification of proteins and/or polypeptides using glycosyl sulfinates as raw materials. The method has some advantages, such as readily available reaction materials, mild reaction conditions, short reaction time, controllable reaction process, and high yield and purity of glycosylation-modified proteins and/or polypeptides.

The glycosylation modification method of the present invention is not only applicable to proteins and/or polypeptides containing disulfide bonds, but also to proteins and/or polypeptides containing sulfhydryls, and even to proteins and/or polypeptides containing both disulfide bonds and sulfhydryls, with broad application prospects.

In the present invention, it is first disclosed that when the method of the present invention is used for glycosylation modification of proteins and/or peptides containing both disulfide bonds and sulfhydryls, the glycosylation groups first modify the sulfhydryls in the proteins and/or polypeptides, and then modify the disulfide bonds in the proteins and/or polypeptides. The method of the present invention can be used to perform process controllable and multiple glycosylation modifications on proteins and/or polypeptides containing both disulfide bonds and sulfhydryls, and thus has broad application prospects.

Obviously, based on the above content of the present invention, according to the common technical knowledge and the conventional means in the field, other various modifications, alternations, or changes can further be made, without department from the above basic technical spirits.

By following description of specific examples, the above content of the present invention is further illustrated. But it should not be construed that the scope of the above subject matter of the present invention is limited to the following examples. The techniques realized based on the above content of the present invention are all within the scope of the present invention.

### DESCRIPTION OF FIGURES

FIG. 1. Schemes of the reaction process of the method for glycosylation modification of proteins and/or polypeptides according to the present invention.

FIG. 2A. Reaction scheme in Example 1.

FIG. 2B. The mass spectrum of the antibody conjugate obtained in Example 1.

FIG. 3. The mass spectrum of the antibody conjugate obtained in Example 2.

FIG. 4. The mass spectrum of polypeptides obtained in Example 5 by single substitution of sulfhydryl at position 1 with xylosyl.

FIG. 5. The mass spectrum of polypeptides obtained in Example 5 by substitution with xylosyl at position 1 as well as with glucosyl at positions 2 and 9.

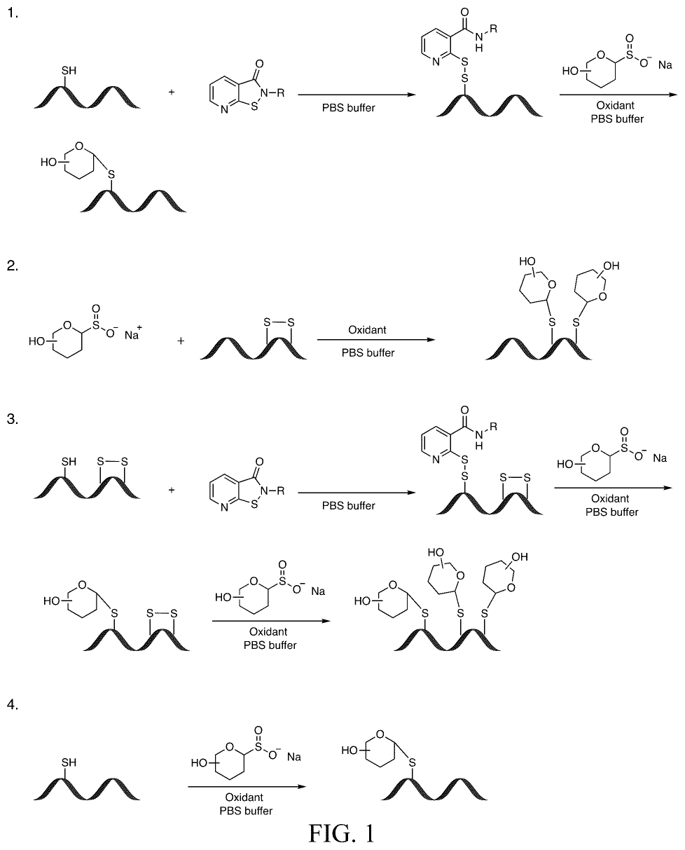

### EXAMPLES

The starting materials and equipment used in the present invention are all known products, which are obtained by purchasing those commercially available.

Glycosyl sulfinates were prepared according to the following procedures:

### I. Preparation of Glycosyl Sulfinate 1

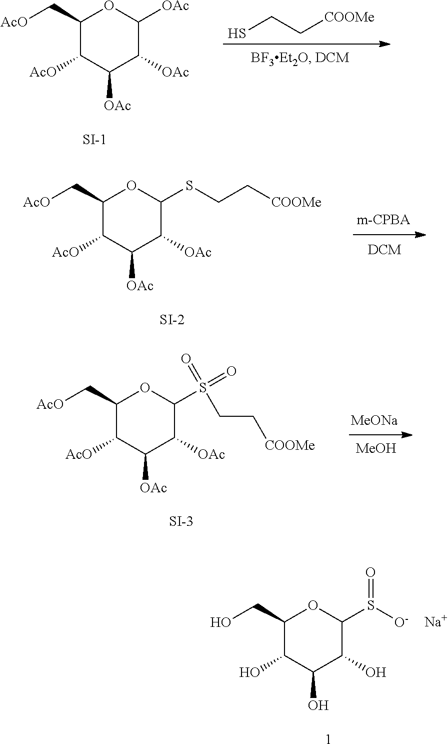

Specific procedures were as follows:

Step 1: To a 100 mL round bottom flask containing SI-1 (3.9 g, 10 mmol, 1.0 equiv) and 25 mL of CH₂Cl₂, were added methyl 3-mercaptopropionate (1.3 mL, 12 mmol, 1.2 equiv) and BF₃·Et₂O (2.5 mL, 20 mmol, 2.0 equiv) sequentially. The reaction solution was stirred at room temperature for 1 h, until SI-1 was completely disappeared by TLC detection, and then washed with saturated NaHCO₃ aqueous solution to be neutral. The organic layers were separated, washed with saline, dried over anhydrous Na₂SO₄, and then concentrated to obtain SI-2, which could be directly used for the next step without purification.

Step 2: SI-2 was dissolved in 20 mL of CH₂Cl₂ and then cooled at 0° C. m-CPBA (m-chloroperoxybenzoic acid, 6 g, 30 mmol, 3 equiv) was slowly added to the reaction solution under stirring. The mixed solution was stirred at room temperature for 1 h and filtered. The filtrate was washed with saturated NaHCO₃ solution until neutral, dried over anhydrous Na₂SO₄, and concentrated. Methyl tert-butyl ether was added to precipitate the solid, which was collected by filtration to obtain SI-3 as white solid.

Step 3: SI-3 was dissolved in 20 mL of MeOH at 0° C., to which was added MeONa (540 mg, 10 mmol, 1.0 equiv), and then the reaction was stirred at 0° C. for 2 h. TLC detection indicated that SI-3 was completely consumed before concentration. The residue was washed with absolute ethanol, and then the resultant solution was filtered to obtain white solid, namely glycosyl sulfinate 1. The total yield for three steps was 85%.

### II. Preparation of Glycosyl Sulfinates 2-20

Glycosyl sulfinate 2-20 were prepared separately by referring to the above method for preparing glycosyl sulfinate 1, with the only difference lying in that the raw material SI-1 was substituted with the corresponding starting materials, respectively.

The structure and characterization of glycosyl sulfinate 1-20 are shown in Table 1. The total yield for three steps and the purity of sodium sulfinate glycosyl donors 1-20 are shown in Table 2.

TABLE 1
The structure and characterization of glycosyl sulfinate 1-20.

| | | Starting materials | Structural |
|---|---|---|---|
| No. | Structure | corresponding to SI-1 | characterization |
| 1 | 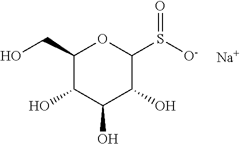 [SMILES: O=S([O-])C1O[C@H](CO)[C@@H](O)[C@H](O)[C@H]1O ; [Na+]] | 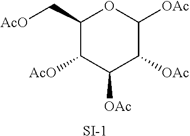 | ¹H NMR (400 MHz, CD₃OD) δ 3.85 (d, J = 12.9 Hz, 1H), 3.73 (t, J = 9.3 Hz, 1H), 3.66 (dd, J = 12.0, 5.2 Hz, 1H), 3.43 (t, J = 8.4 Hz, 1H), 3.35 (d, J = 9.7 Hz, 1H), 3.30 (d, J = 5.3 Hz, 2H); ¹³C NMR (101 MHz, D₂O) δ 92.68, 79.99, 77.10, 69.76, 69.16, 60.94. |
| 2 | 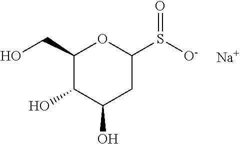 [SMILES: O=S([O-])C1C[C@@H](O)[C@H](O)[C@@H](CO)O1 ; [Na+]] | 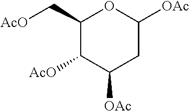 [SMILES: CC(=O)OC[C@H]1OC(OC(C)=O)C[C@@H](OC(C)=O)[C@@H]1OC(C)=O] | ¹³C NMR (101 MHz, D₂O) δ 96.29, 76.96, 70.67, 68.55, 60.78, 31.38. |
| 3 | 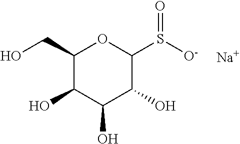 [SMILES: O=S([O-])C1O[C@H](CO)[C@H](O)[C@H](O)[C@H]1O ; [Na+]] | 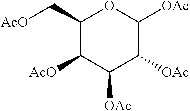 [SMILES: CC(=O)OC[C@H]1OC(OC(C)=O)[C@H](OC(C)=O)[C@@H](OC(C)=O)[C@H]1OC(C)=O] | ¹H NMR (400 MHz, CD₃OD) δ 3.99 (t, J = 9.5 Hz, 1H), 3.83 (dd, J = 11.7, 7.7 Hz, 1H), 3.78 (d, J = 3.3 Hz, 1H), 3.66-3.60 (m, 1H), 3.58 (q, J = 3.9 Hz, 1H), 3.53 (dd, J = 9.4, 3.4 Hz, 1H), 3.34 (d, J = 3.5 Hz, 1H). ¹³C NMR (101 MHz, D₂O) δ 93.72, 79.49, 73.96, 69.08, 67.06, 61.60. |
| 4 | 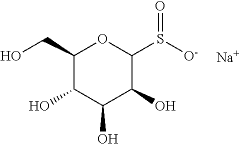 [SMILES: O=S([O-])C1O[C@H](CO)[C@@H](O)[C@H](O)[C@@H]1O ; [Na+]] | 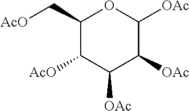 [SMILES: CC(=O)OC[C@H]1OC(OC(C)=O)[C@@H](OC(C)=O)[C@@H](OC(C)=O)[C@@H]1OC(C)=O] | ¹H NMR (400 MHz, D₂O) δ 4.22 (d, J = 3.0 Hz, 1H), 3.78 (m, 2H), 3.71-3.62 (m, 3H), 3.50 (d, J = 1.3 Hz, 1H); ¹³C NMR (101 MHz, D₂O) δ 100.65, 77.55, 71.25, 67.94, 66.20, 1.06 |
| 5 | 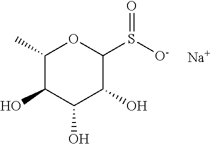 | 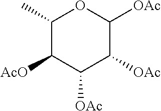 [SMILES: CC(=O)OC1O[C@@H](C)[C@H](OC(C)=O)[C@@H](OC(C)=O)[C@H]1OC(C)=O] | ¹H NMR (400 MHz, D₂O) δ 4.33-4.29 (m, 1H), 4.20-4.10 (m, 1H), 3.85-3.76 (m, 2H), 3.52-3.45 (m, 1H), 1.31 (d, J = 6.1, 3H). ¹³C NMR (101 MHz, D₂O) δ 90.32, 71.66, 69.90, 68.21, 57.41, 16.76. |
| 6 | 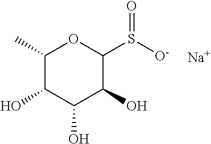 | 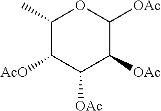 [SMILES: CC(=O)OC1O[C@@H](C)[C@@H](OC(C)=O)[C@@H](OC(C)=O)[C@@H]1OC(C)=O] | ¹H NMR (400 MHz, D₂O) δ 3.86-3.80 (m, 1H), 3.72 (d, J = 6.3 Hz, 1H), 3.62 (dt, J = 10.6, 2.9 Hz, 1H), 3.56 (td, J = 7.1, 1.9 Hz, 1H), 3.39 (dd, J = 9.7, 1.9 Hz, 1H), 1.22-1.17 (m, 3H); ¹³C NMR (101 MHz, D₂O) δ 93.75, 75.19, 74.10, 71.61, 66.57, 16.78. |
| 7 | 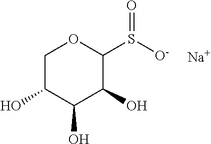 [SMILES: O=S([O-])C1OC[C@@H](O)[C@H](O)[C@@H]1O ; [Na+]] | 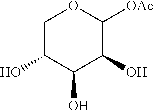 [SMILES: CC(=O)OC1OC[C@@H](O)[C@H](O)[C@@H]1O] | ¹³C NMR (101 MHz, D₂O) δ 99.50, 73.85, 70.79, 69.85, 68.38. |
| 8 | 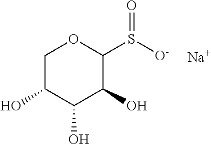 [SMILES: O=S([O-])C1OC[C@@H](O)[C@@H](O)[C@@H]1O ; [Na+]] | 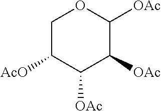 [SMILES: CC(=O)OC1OC[C@@H](OC(C)=O)[C@@H](OC(C)=O)[C@@H]1OC(C)=O] | Mixture of a and b□¹H NMR (400 MHz, D₂O) δ 4.37 (td, J = 3.9, 1.3 Hz, 0.5 H), 4.03-3.83 (m, 3H), 3.75-3.54 (m, 2.5H); ¹³C NMR (101 MHz, D₂O) δ 96.71, 72.41, 71.81, 70.31, 68.44. |
| 9 | 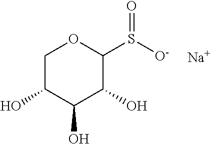 [SMILES: O=S([O-])C1OC[C@@H](O)[C@H](O)[C@H]1O ; [Na+]] | 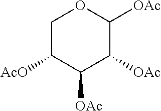 [SMILES: CC(=O)OC1OC[C@@H](OC(C)=O)[C@H](OC(C)=O)[C@H]1OC(C)=O] | ¹H NMR (400 MHz, D₂O) δ 3.98 (dd, J = 11.1, 5.3 Hz, 1H), 3.60 (t, J = 9.2 Hz, 1H), 3.52 (td, J = 9.7, 5.2 Hz, 1H), 3.42 (t, J = 9.2 Hz, 2H), 3.22 (t, J = 10.8 Hz, 1H); ¹³C NMR (101 MHz, D₂O) δ 93.61, 77.12, 69.54, 69.15, 68.86. |
| 10 | 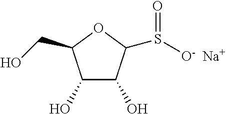 [SMILES: O=S([O-])C1O[C@H](CO)[C@@H](O)[C@H]1O ; [Na+]] | 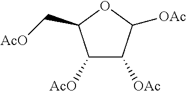 [SMILES: CC(=O)OC[C@H]1OC(OC(C)=O)[C@H](OC(C)=O)[C@@H]1OC(C)=O] | Mixture of α:β = 5:4, ¹H NMR (400 MHz, D₂O): δ 4.43 (t, J = 4.2 Hz, 0.43H), 4.29 (dd, J = 4.7, 2.1 Hz, 1H), 4.05 (dd, J = 8.4, 4.5 Hz, 0.55H), 4.00-3.91 (m, 2.46H), 3.88-3.68 (m, 2.94H), and 3.58 (td, J = 12.4, 4.7 Hz, 1.74H); ¹³C NMR (101 MHz, D₂O): δ 102.18, 100.63, 83.01, 82.53, 71.53, 71.30, 71.16, 70.70, 61.70, 60.76. |
| 11 | 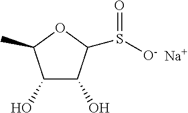 | 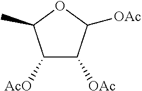 [SMILES: CC(=O)OC1O[C@H](C)[C@@H](OC(C)=O)[C@H]1OC(C)=O] | ¹H NMR (400 MHz, Methanol-d₄) δ 4.28 (d, J = 5.0 Hz, 1H), 3.96 (t, J = 6.3 Hz, 1H), 3.81 (t, J = 1.8 Hz, 1H), 3.73-3.65 (m, 1H), 1.21 (d, J = 6.3 Hz, 3H); ¹³C NMR (101 MHz, D₂O) δ 101.85, 78.88, 76.35, 70.51, 17.67. |
| 12 | 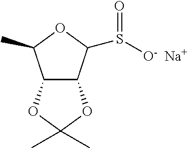 | 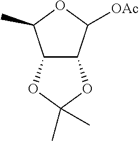 | ¹H NMR (400 MHz, CD₃OD) δ 5.01 (t, J = 4.5 Hz, 1H), 4.45 (d, J = 6.4 Hz, 1H), 4.20 (q, J = 7.1 Hz, 1H), 3.62 (dd, J = 4.4, 1.7 Hz, 1H), 1.46 (s, 3H), 1.30 (s, 3H), 1.15 (d, J = 5.1 Hz, 3H). |
| 13 | 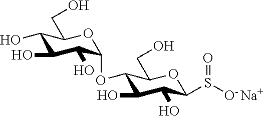 | 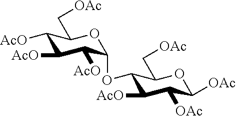 | ¹H NMR (400 MHz, D₂O) δ 5.32 (d, J = 3.8 Hz, 1H), 3.86 (d, J = 11.7 Hz, 1H), 3.81-3.44 (m, 11H), 3.33 (t, J = 9.4 Hz, 1H); ¹³C NMR (101 MHz, D₂O) δ 99.67, 92.47, 78.56, 77.51, 76.44, 72.83, 72.66, 71.73, 69.62, 69.30, 60.94, 60.43. |
| 14 | 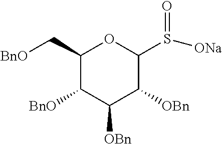 | 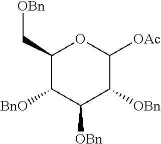 [SMILES: CC(=O)OC1O[C@H](COCc2ccccc2)[C@@H](OCc2ccccc2)[C@H](OCc2ccccc2)[C@H]1OCc1ccccc1] | ¹H NMR (400 MHz, CDCl₃) δ 7.36-7.27 (m, 14H), 7.26-7.22 (m, 4H), 7.15-7.06 (m, 2H), 4.93 (d, J = 11.1 Hz, 1H), 4.89 (d, J = 11.1 Hz, 1H), 4.81 (d, J = 10.7 Hz, 1H), 4.76 (d, J = 11.1 Hz, 1H), 4.68 (d, J = 10.6 Hz, 1H), 4.44 (d, J = 11.3 Hz, 1H), 4.41 (d, J = 11.1 Hz, 1H), 4.36 (d, J = 11.2 Hz, 1H), 4.11 (d, J = 9.7 Hz, 1H), 3.85 (t, J = 8.8 Hz, 1H), 3.77 (t, J = 9.4 Hz, 1H), 3.67 (ddt, J = 7.9, 4.8, 2.9 Hz, 2H), 3.60- 3.54 (m, 1H), 3.51 (dd, J = 10.7, 5.4 Hz, 1H); ¹³C |
| | | | NMR (101 MHz, CDCl₃): δ 161.1, 160.7, 160.2, |
| | | | 159.8, 137.0, 136.5, 136.3, 135.3, 129.0, 128.9, |
| | | | 128.7, 128.7, 128.6, 128.6, 128.5, 128.4, 128.3, |
| | | | 128.0, 118.7, 115.9, 113.1, 110.2, 92.0, 85.7, 78.4, |
| | | | 77.3, 77.0, 76.8, 76.7, 76.1, 75.6, 75.4, 73.8, 67.9 |
| 15 | 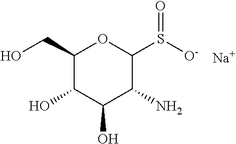 [SMILES: N[C@H]1C(S(=O)[O-])O[C@H](CO)[C@@H](O)[C@@H]1O ; [Na+]] | 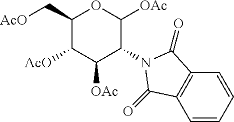 [SMILES: CC(=O)OC[C@H]1OC(OC(C)=O)[C@H](N2C(=O)c3ccccc3C2=O)[C@@H](OC(C)=O)[C@@H]1OC(C)=O] | ¹H NMR (400 MHz, D₂O) δ 3.92 (d, J = 13.0 Hz, 1H), 3.76 (dd, J = 12.5, 4.9 Hz, 1H), 3.71 (d, J = 10.3 Hz, 1H), 3.68-3.60 (m, 1H), 3.48 (ddd, J = 22.8, 17.9, 9.0 Hz, 2H), 3.39-3.31 (m, 1H). |
| 16 | 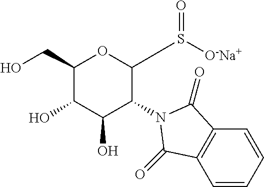 | 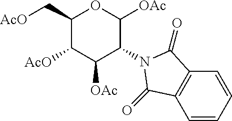 [SMILES: CC(=O)OC[C@H]1OC(OC(C)=O)[C@H](N2C(=O)c3ccccc3C2=O)[C@@H](OC(C)=O)[C@@H]1OC(C)=O] | ¹H NMR (400 MHz, MeOD) δ 7.84 (s, 2H), 7.77 (dd, J = 5.5, 3.1 Hz, 2H), 4.29 (dd, J = 6.7, 2.8 Hz, 2H), 4.13 (dd, J = 7.2, 3.3 Hz, 1H), 3.93 (dd, J = 12.1, 2.1 Hz, 1H), 3.73-3.67 (m, 1H), 3.46 (ddd, J = 9.3, 6.7, 2.2 Hz, 1H), 3.34 (s, 1H). |
| 17 | 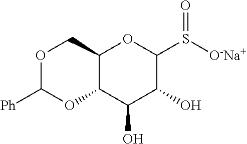 | 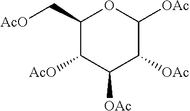 [SMILES: CC(=O)OC[C@H]1OC(OC(C)=O)[C@H](OC(C)=O)[C@@H](OC(C)=O)[C@@H]1OC(C)=O] | ¹H NMR (400 MHz, MeOD) δ 7.48 (dd, J = 6.8, 3.1 Hz, 2H), 7.41 (d, J = 2.5 Hz, 2H), 7.40-7.39 (m, 1H), 5.68 (s, 1H), 4.31 (td, 1H), 3.82 (t, J = 10.2 Hz, 1H), 3.78-3.72 (m, 2H), 3.60 (dqd, J = 9.3, 7.8, 7.3, 3.6 Hz, 3H). |
| 18 | 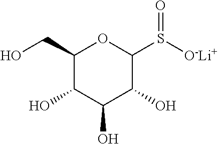 | 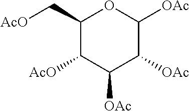 [SMILES: CC(=O)OC[C@H]1OC(OC(C)=O)[C@H](OC(C)=O)[C@@H](OC(C)=O)[C@@H]1OC(C)=O] | ¹H NMR (400 MHz, CD₃OD) δ 3.85 (d, J = 12.9 Hz, 1H), 3.73 (t, J = 9.3 Hz, 1H), 3.66 (dd, J = 12.0, 5.2 Hz, 1H), 3.43 (t, J = 8.4 Hz, 1H), 3.35 (d, J = 9.7 Hz, 1H), 3.30 (d, J = 5.3 Hz, 2H) |
| 19 | 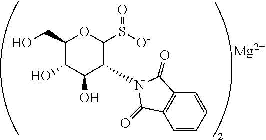 | 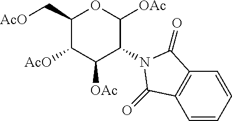 [SMILES: CC(=O)OC[C@H]1OC(OC(C)=O)[C@H](N2C(=O)c3ccccc3C2=O)[C@@H](OC(C)=O)[C@@H]1OC(C)=O] | ¹H NMR (400 MHz, CD₃OD) δ 7.83-7.76 (m, 1H), 7.55-7.50 (m, 1H), 7.47 (d, J = 4.5 Hz, 2H), 4.16 (t, J = 10.3 Hz, 1H), 3.90 (d, J = 12.1 Hz, 1H), 3.79- 3.75 (m, 1H), 3.74-3.67 (m, 11H), 3.36-3.32 (m, 1H). |
| 20 | 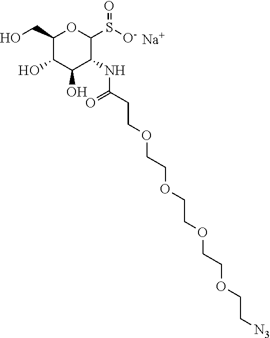 [SMILES: [N-]=[N+]=NCCOCCOCCOCCOCCC(=O)N[C@H]1C(S(=O)[O-])O[C@H](CO)[C@@H](O)[C@@H]1O ; [Na+]] | 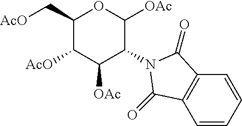 [SMILES: CC(=O)OC[C@H]1OC(OC(C)=O)[C@H](N2C(=O)c3ccccc3C2=O)[C@@H](OC(C)=O)[C@@H]1OC(C)=O] | ¹H NMR (400 MHz, D₂O) δ 3.94 (t, J = 10.3 Hz, 2H), 3.86-3.76 (m, 3H), 3.76-3.67 (m, 14H), 3.61 (d, J = 10.6 Hz, 2H), 3.54-3.50 (m, 2H), 3.49-3.42 (m, 2H), 2.58 (t, J = 6.2 Hz, 2H). |

TABLE 2
The total yield for three steps and the purity of final product glycosyl sulfinate 1-20.

| | | Total yield for | |
|---|---|---|---|
| No. | Structure | three steps | Purity |
| 1 | 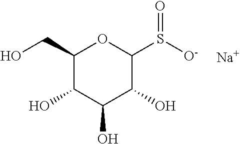 [SMILES: O=S([O-])C1O[C@H](CO)[C@@H](O)[C@H](O)[C@H]1O ; [Na+]] | 85% | 99% |
| 2 | 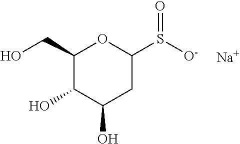 [SMILES: O=S([O-])C1C[C@@H](O)[C@H](O)[C@@H](CO)O1 ; [Na+]] | 77% | 95% |
| 3 | 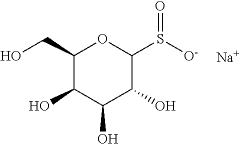 [SMILES: O=S([O-])C1O[C@H](CO)[C@H](O)[C@H](O)[C@H]1O ; [Na+]] | 87% | 98% |
| 4 | 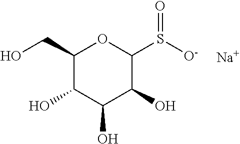 [SMILES: O=S([O-])C1O[C@H](CO)[C@@H](O)[C@H](O)[C@@H]1O ; [Na+]] | 75% | 98% |
| 5 | 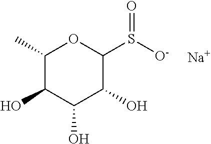 | 67% | 90% |
| 6 | 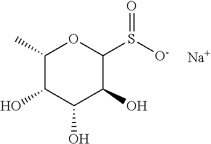 | 70% | 90% |
| 7 | 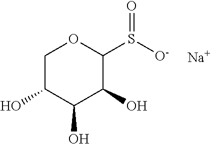 [SMILES: O=S([O-])C1OC[C@@H](O)[C@H](O)[C@@H]1O ; [Na+]] | 70% | 90% |
| 8 | 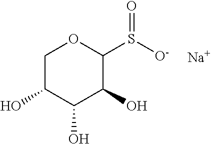 [SMILES: O=S([O-])C1OC[C@@H](O)[C@@H](O)[C@@H]1O ; [Na+]] | 70% | 90% |
| 9 | 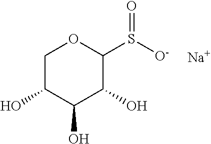 [SMILES: O=S([O-])C1OC[C@@H](O)[C@H](O)[C@H]1O ; [Na+]] | 70% | 90% |
| 10 | 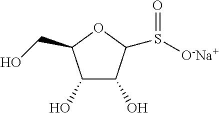 | 65% | 85% |
| 11 | 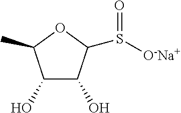 | 85% | 95% |
| 12 | 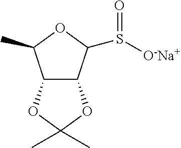 | 60% | 90% |
| 13 | 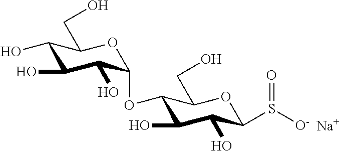 | 75% | 95% |
| 14 | 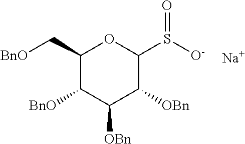 [SMILES: O=S([O-])C1O[C@H](COCc2ccccc2)[C@@H](OCc2ccccc2)[C@H](OCc2ccccc2)[C@H]1OCc1ccccc1 ; [Na+]] | 85% | 98% |
| 15 | 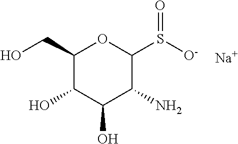 [SMILES: N[C@H]1C(S(=O)[O-])O[C@H](CO)[C@@H](O)[C@@H]1O ; [Na+]] | 82% | 98% |
| 16 | 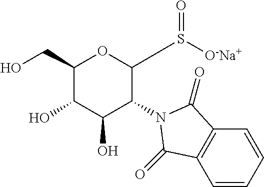 | 85% | 98% |
| 17 | 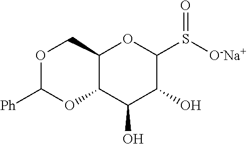 | 70% | 97% |
| 18 | 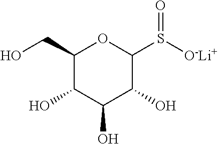 | 85% | 98% |
| 19 | 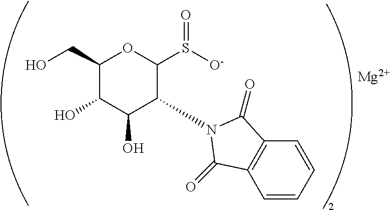 | 70% | 85% |
| 20 | 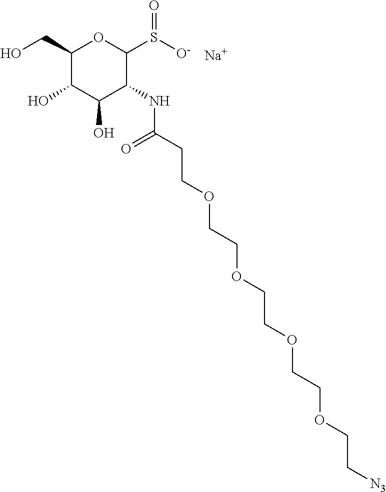 [SMILES: [N-]=[N+]=NCCOCCOCCOCCOCCC(=O)N[C@H]1C(S(=O)[O-])O[C@H](CO)[C@@H](O)[C@@H]1O ; [Na+]] | 57% | 98% |

### Example 1: Method for Glycosylation Modification of Antibodies Containing Disulfide Bonds

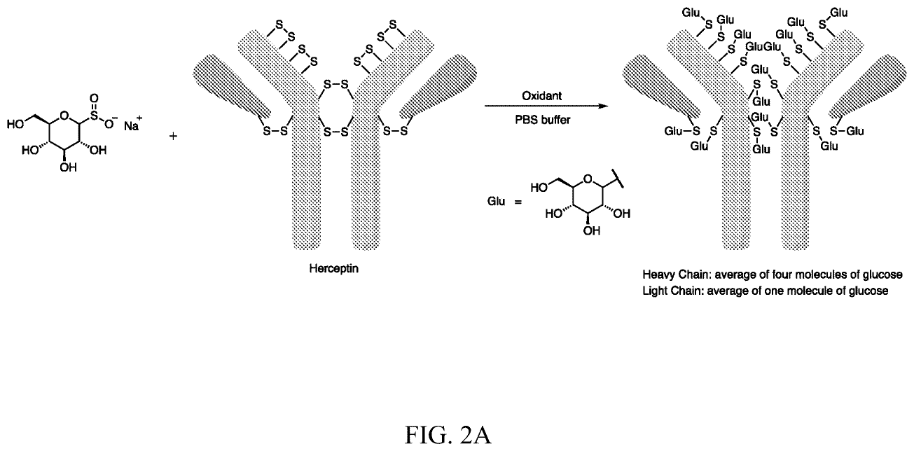

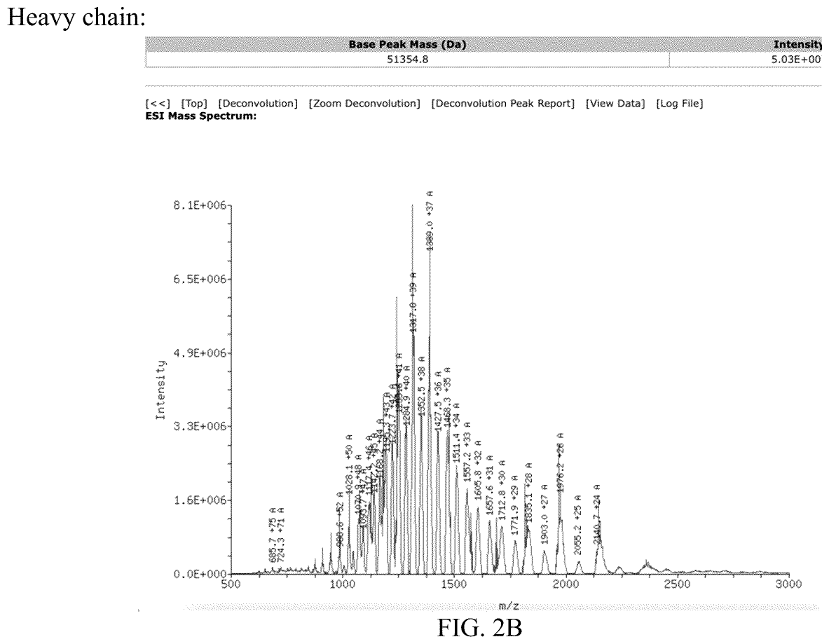

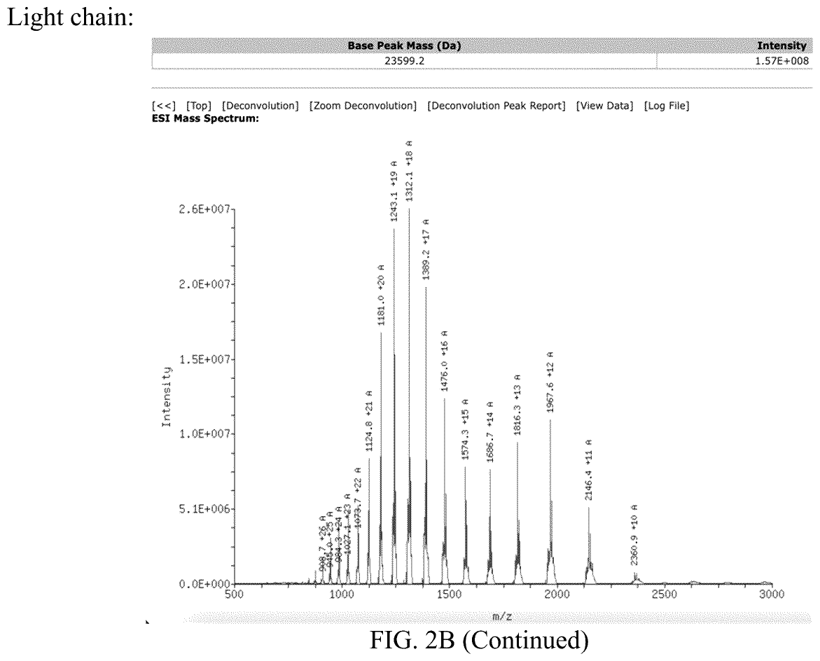

According to the reaction scheme of FIG. 2A, the antibody underwent glycosylation modification to synthesize antibody conjugates. The specific procedures were as follows: sodium glycosyl sulfinate (4 μmol), Herceptin antibody containing disulfide bonds from Roche Pharmaceuticals (0.01 μmol), tert-butyl hydroperoxide (4 μmol), and PBS buffer (0.1 mL) were weighed, transferred into a vial with a spiral cap, and then mixed. The vial was filled with N₂ and sealed with a Teflon cap. The mixture was allowed to stand at room temperature for 1 h before the reaction was completed.

The reaction solution was separated using a semipermeable membrane (with molecular weight cut-off of 3 kDa) to obtain antibody conjugates as pure α-configuration, with a yield of 95% and a purity of >98%. According to primary and secondary MS analysis, antibody conjugates have an average of four glucose molecules attached to each heavy chain and one glucose molecule attached to each light chain.

The sequence characterization was as follows:

Heavy Chain with an Average of Three Glucose Molecules Attached to Each Heavy Chain):

(SEQ ID NO. 1)
EVQLVESGGGLVQPGGSLR+LSCAASGFNIK+DTYIHWVR+QAPGK+
GLEWVAR+IYPTNGYTR+YADSVK+GR+FTISADTSK+NTAYLQMNS
LR+AEDTAVYYCSR+WGGDGFYAMDYWGQGTLVTVSSASTK+GPSVF
PLAPSSK+STSGGTAALGC(-Glu)LVK+DYFPEPVTVSWNSGALTS
GVHTFPAVLQSSGLYSLSSVVTVPSSSLGTQTYICNVNHK+PSNTK+
VDK+K+VEPK+SCDK+THTC(-Glu)PPC(-Glu)PAPELLGGPSVF
LFPPK+PK+DTLMISR+TPEVTCVVVDVSHEDPEVK+FNWYVDGVEV
HNAK+TK+PR+EEQYNSTYR+VVSVLTVLHQDWLNGK+EYK+CK+VS
NK+ALPAPIEK+TISK+AK+GQPR+EPQVYTLPPSR+EEMTK+NQVS
LTCLVK+GFYPSDIAVEWESNGQPENNYK+TTPPVLDSDGSFFLYSK+
LTVDK+SR+WQQGNVFSCSVMHEALHNHYTQK+SLSLSPG.

Light Chain (with an Average of One Glucose Molecule Attached to Each Light Chain):

(SEQ ID NO. 2)
DIQMTQSPSSLSASVGDR+VTITCR+ASQDVNTAVAWYQQK+PGK+AP
K+LLIYSASFLYSGVPSR+FSGSR+SGTDFTLTISSLQPEDFATYYCQ
QHYTTPPTFGQGTK+VEIK+R+TVAAPSVFIFPPSDEQLK+SGTASVV
CLLNNFYPR+EAK+VQWK+VDNALQSGNSQESVTEQDSK+DSTYSLSS
TLTLSK+ADYEK+HK+VYACEVTHQGLSSPVTK+SFNR+GEC(-Glu)

The MS characterization is shown in FIG. 2B.

### Example 2: Method for Glycosylation Modification of Affibody Containing a Sulfhydryl Group

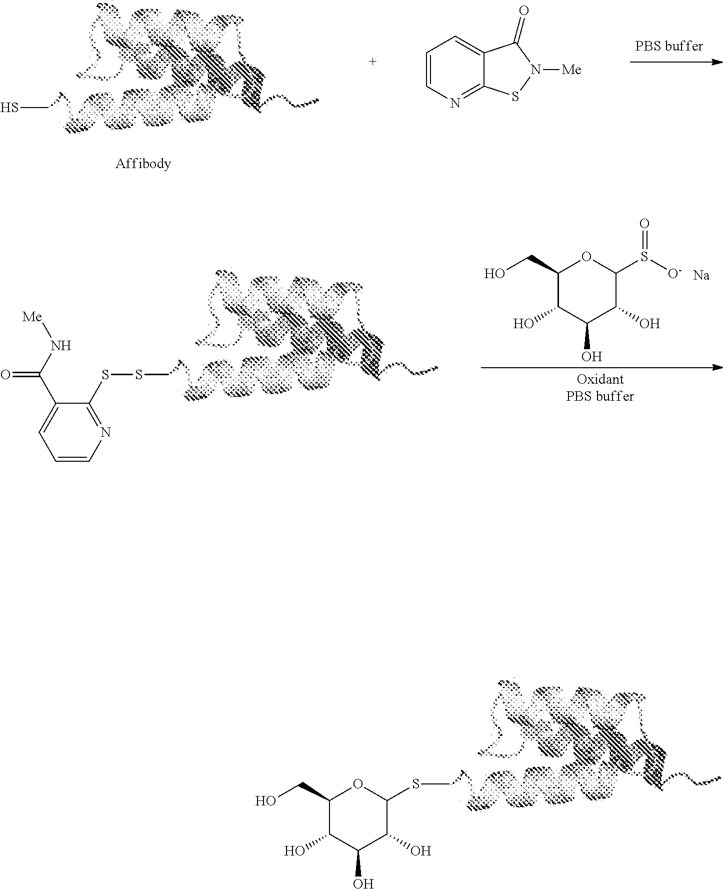

### 1. Preparation of Affibody Containing a Sulfhydryl Group

The gene fragment with a nucleotide sequence of 5′-ATGGGCAGCAGCCATCATCAT CATCATCACAGCAGCGGCGAAAACCTGTATTTTCAGGGCCATATGGTTGATAACAAATT TAACAAAGAAATGCGCAACGCATATTGGGAAATTGCACTGCTGCCGAATCTGAATAATC AGCAGAAACGTGCGTTTATTCGTAGCCTGTATGATGATCCGAGTCAGAGCGCAAACCTG CTGGCAGAAGCAAAAAAACTGAATGATGCACAGGCACCGAAATGCtaG-3′ (SEQ ID NO.3) was inserted into a plasmid of Escherichia coli to express Affibody containing sulfhydryl. Then, Western Blot was performed for exposure, and then the bacterial cells were suspended, and broken by supersonic technique. The supernatant was separated, and subjected to Ni-affinity chromatography (eluted with binding buffer, and the binding buffer was composed of 50 mM Tris and 150 mM NaCl, pH=7.4), followed by protein gel column chromatography, which was continuously eluted with 100 mL wash buffer (the wash buffer was composed of 50 mM Tris, 150 mM NaCl, and 20 mM imidazole (imidazole), pH=7.4). Then, the eluant was collected, concentrated, and purified to obtain sulfhydryl-containing Affibody, with a sequence of

(SEQ ID NO. 4)
MGSSHHHHHHSSGENLYFQGHMVDNKFNKEMRNAYWEIALLPN
LNNQQKRAFIRSLYDDPSQSANLLAEAKKLNDAQAPKC.

### 2. Glycosylation Modification of Affibody Containing a Sulfhydryl Group

Firstly, Affibody (0.01 μmol) containing sulfhydryl was mixed with 2-methylisothiazolo[4,5-b]pyridin-3(2H)-one (2 μmol) and PBS buffer (0.1 mL) in a vial with a spiral cap. After standing at room temperature for 10 min, sodium glycosyl sulfinate (4 μmol) and tert-butyl hydroperoxide (4 μmol) were weighed, transferred into the vial with a spiral cap, and then mixed. The vial was filled with N₂ and sealed with a Teflon cap. After the mixture was allowed to stand at room temperature for 1 h, the reaction was completed.

The reaction solution was separated using a semipermeable membrane (with molecular weight cut-off of 3 kDa) to obtain antibody conjugates as pure α-configuration, with a yield of 95% and a purity of >98%.

The sequence characterization was as follows:

(SEQ ID NO. 5)
MGSSHHHHHHSSGENLYFQGHMVDNKFNKEMRNAYWEIALLPNL
NNQQKRAFIRSLYDDPSQSANLLAEAKKLNDAQAPKC(-Glu).

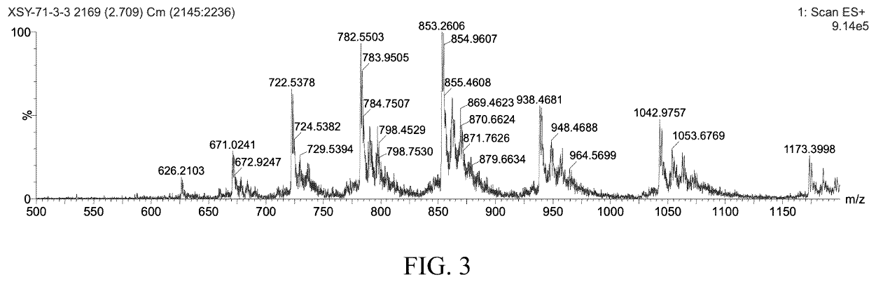

The MS characterization was as follows: [M+11H]/11=853.2606 (see FIG. 3).

### Example 3: Method 1 for Glycosylation Modification of Polypeptides Containing a Sulfhydryl Group

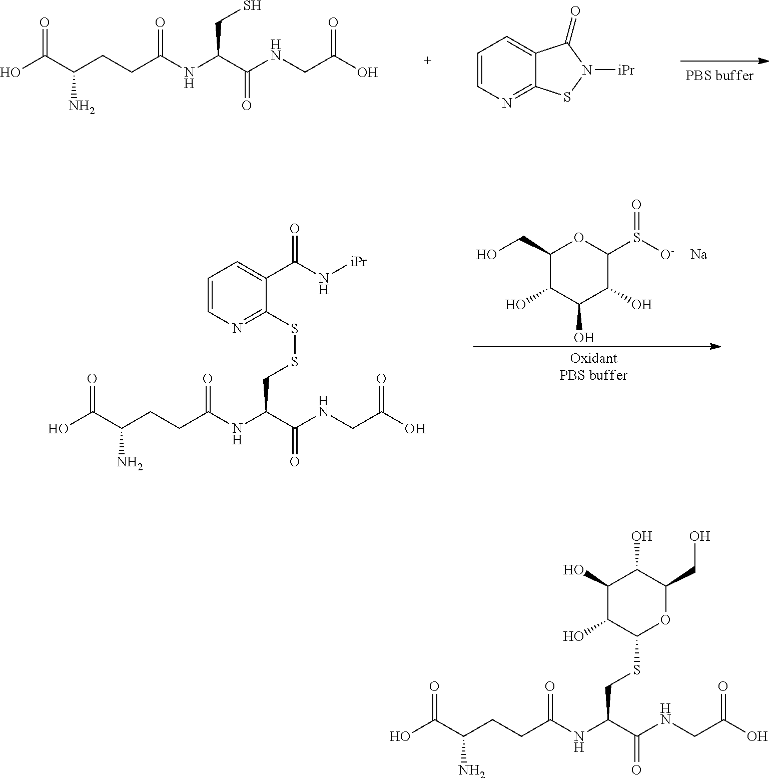

According to the above route, glycosylation modification of polypeptides was carried out to synthesize polypeptide conjugates. The specific procedures were as follows: firstly, reduced glutathione containing sulfhydryl (0.01 mmol) was mixed with 2-isopropylisothiazolo[4,5-b]pyridin-3(2H)-one (0.03 μmol) and PBS buffer (0.1 mL). After standing at room temperature for 10 min, sodium glycosyl sulfinate (0.06 μmol) and tert-butyl hydroperoxide (0.06 μmol) were weighed, transferred into a vial with a spiral cap, and then mixed. The vial was filled with N₂ and sealed with a Teflon cap. The mixture was allowed to stand at room temperature for 1 h, and then the reaction was completed.

The reaction solution was separated over reversed-phase column (gradient elution, using a mixed solution of acetonitrile and water as the eluent, wherein the volume percentage of acetonitrile was 5%-95%) to obtain polypeptide conjugates as pure α-configuration, with a yield of 95% and a purity of >98%. The structural characterization was as follows:

¹H NMR (400 MHz, D₂O) δ 5.50 (d, J=5.5 Hz, 1H), 4.64 (dd, J=8.3, 5.1 Hz, 1H), 4.04-3.94 (m, 4H), 3.87-3.77 (m, 3H), 3.55 (t, J=9.4 Hz, 1H), 3.44 (t, J=9.3 Hz, 1H), 3.16-3.04 (m, 2H), 2.64-2.54 (m, 2H), 2.27-2.19 (m, 2H).

¹³C NMR (101 MHz, D₂O) δ 174.42, 172.94, 172.69, 172.21, 87.10, 73.47, 72.53, 70.91, 69.36, 60.28, 53.92, 52.72, 41.12, 32.44, 30.99, 25.63.

¹⁹F NMR (376 MHz, D₂O) δ −75.61.

### Example 4: Method 2 for Glycosylation Modification of Polypeptides Containing a Sulfhydryl Group

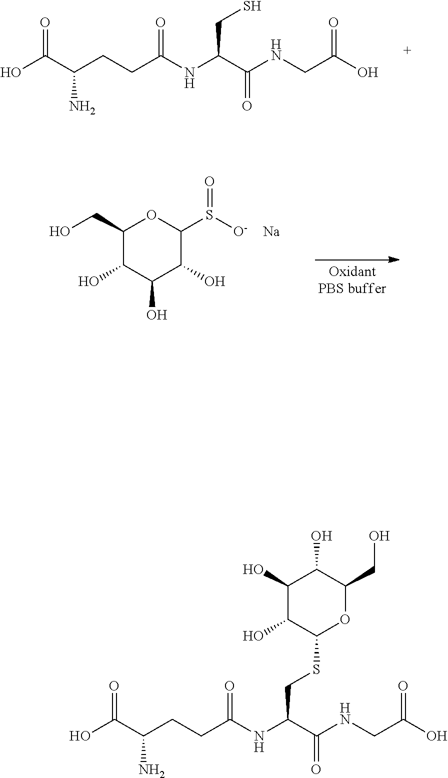

According to the above route, glycosylation modification of polypeptides was carried out to synthesize polypeptide conjugates. The specific procedures were as follows: reduced glutathione containing sulfhydryl (0.01 mmol), sodium glycosyl sulfinate (0.06 μmol), tert-butyl hydroperoxide (0.06 μmol), and PBS buffer (1 mL) were weighed, transferred into a vial with a spiral cap, and then mixed. The vial was filled with N₂ and sealed with a Teflon cap. The mixture was allowed to stand at room temperature for 1 h before completion of the reaction.

The reaction solution was separated over reversed-phase column (gradient elution, using a mixed solution of acetonitrile and water as the eluent, wherein the volume percentage of acetonitrile was 5%-95%) to obtain polypeptide conjugates as pure α-configuration, with a yield of 50% and a purity of >98%. The structural characterization was as follows:

¹H NMR (400 MHz, D₂O) δ 5.50 (d, J=5.5 Hz, 1H), 4.64 (dd, J=8.3, 5.1 Hz, 1H), 4.04-3.94 (m, 4H), 3.87-3.77 (m, 3H), 3.55 (t, J=9.4 Hz, 1H), 3.44 (t, J=9.3 Hz, 1H), 3.16-3.04 (m, 2H), 2.64-2.54 (m, 2H), 2.27-2.19 (m, 2H).

¹³C NMR (101 MHz, D₂O) δ 174.42, 172.94, 172.69, 172.21, 87.10, 73.47, 72.53, 70.91, 69.36, 60.28, 53.92, 52.72, 41.12, 32.44, 30.99, 25.63.

¹⁹F NMR (376 MHz, D₂O) δ −75.61.

### Example 5: One-Pot Two-Step Method for Preparing Polypeptide Conjugates with Different Glycosylation Modifications

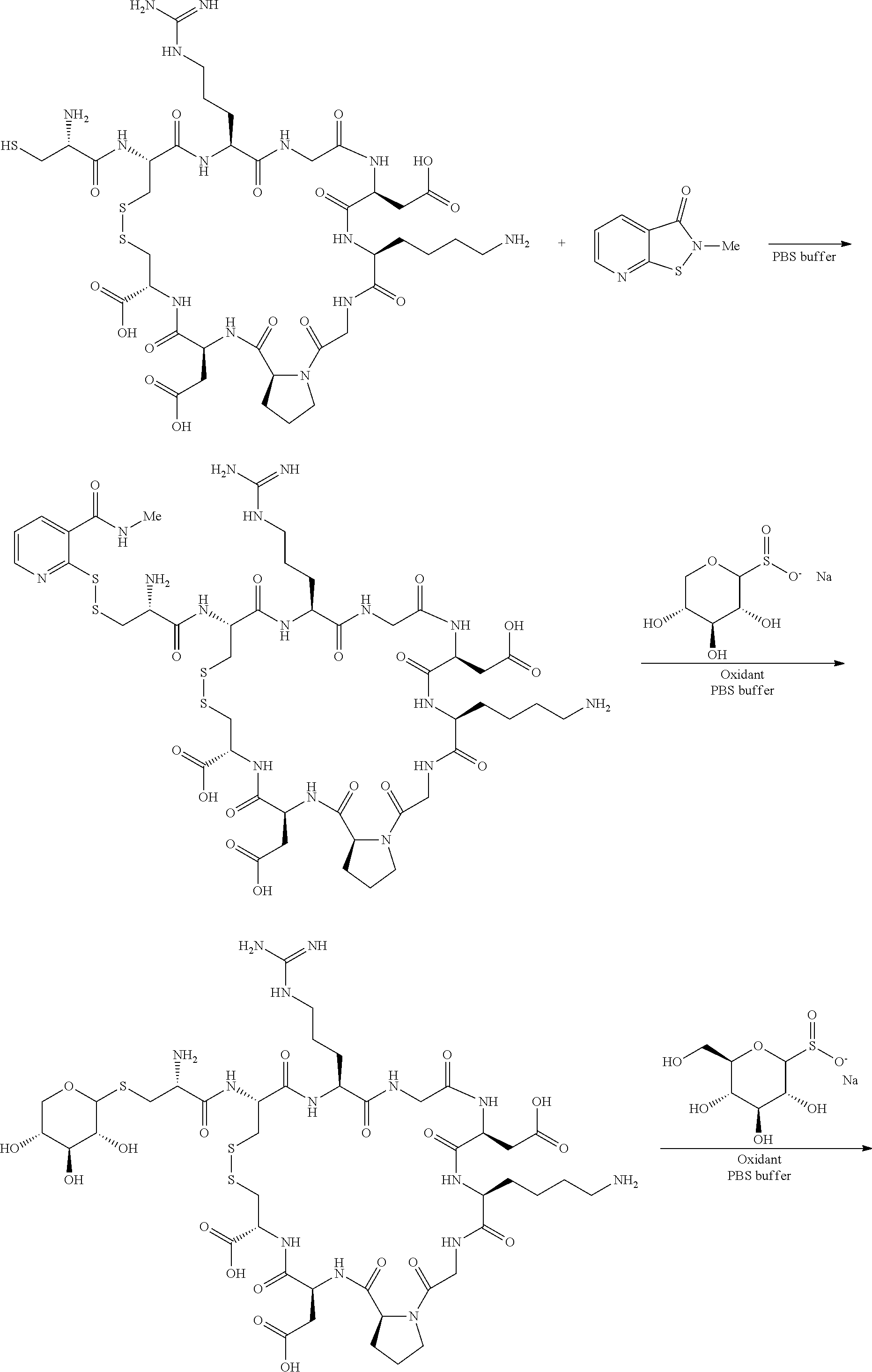

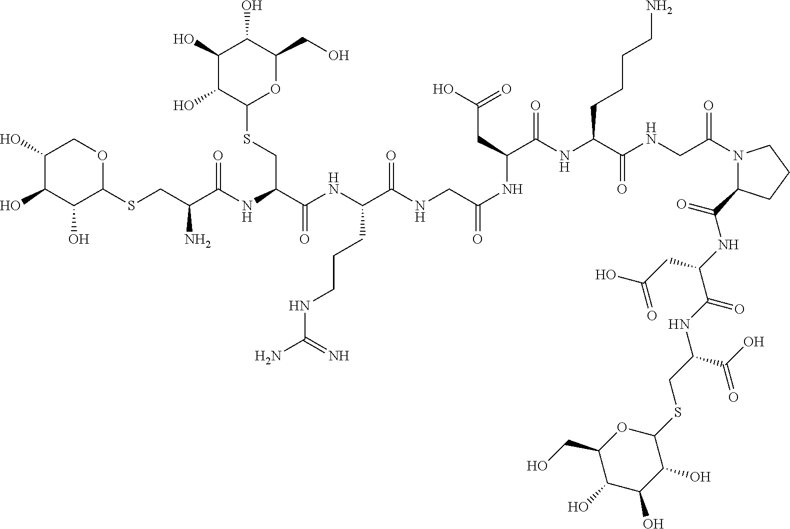 [SMILES: N=C(N)NCCC[C@H](NC(=O)[C@H](CSC1O[C@H](CO)[C@@H](O)[C@H](O)[C@H]1O)NC(=O)[C@@H](N)CSC1OC[C@@H](O)[C@H](O)[C@H]1O)C(=O)NCC(=O)N[C@@H](CC(=O)O)C(=O)N[C@@H](CCCCN)C(=O)NCC(=O)N1CCC[C@H]1C(=O)N[C@@H](CC(=O)O)C(=O)N[C@@H](CSC1O[C@H](CO)[C@@H](O)[C@H](O)[C@H]1O)C(=O)O]

According to the above route, different glycosylation modifications were carried out to synthesize polypeptide conjugates. The specific procedures were as follows: firstly, a polypeptide containing both sulfhydryl and disulfide bonds (the polypeptide sequence was CCRGDKGPDC (SEQ ID NO.6, cysteines at position 2 and position 9 form a disulfide bond, 0.005 mmol) was mixed with 2-methylisothiazolo[4,5-b]pyridin-3(2H)-one (0.006 mmol) and water (1 mL). After standing at room temperature for 10 min, sodium xylosylsulfinate (0.006 mmol) and tert-butyl hydroperoxide (0.008 mmol) were weighed, transferred into a vial with a spiral cap, and then mixed. The vial was filled with N₂ and sealed with a Teflon cap. The mixture was allowed to stand at room temperature for 1 h before completion of the reaction, and then a polypeptide with a single substitution of sulfhydrylxylosyl at position 1 was obtained. Subsequently, sodium glucosylsulfinate (0.01 mmol) and tert-butyl hydroperoxide (0.01 mmol) were weighed, transferred into the same vial, and then mixed. The vial was filled with N₂ and sealed with a Teflon cap. The mixture was stirred at room temperature for 1 h before completion of the reaction.

The reaction solution was separated over reversed-phase column (gradient elution, using a mixed solution of acetonitrile and water as the eluent, wherein the volume percentage of acetonitrile was 5%-95%), to obtain polypeptide substituted with xylosyl at position 1 and glucosyl at positions 2 and 9 as pure α-configuration, with a yield of 95% and a purity of >98%.

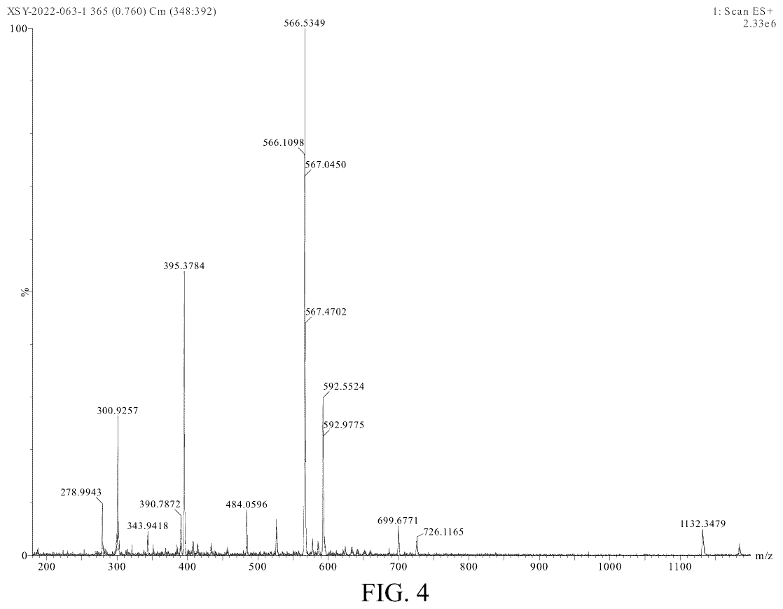

Polypeptide with single substitution of sulfhydrylxylosyl at position 1:
the sequence characterization was as follows:
C(-Xyl)CRGDKGPDC ((SEQ ID NO.7, C at positions 2 and 9 form a disulfide bond);
MS characterization was as follows: [M+2H]/2=592.5524 (see FIG. 4).

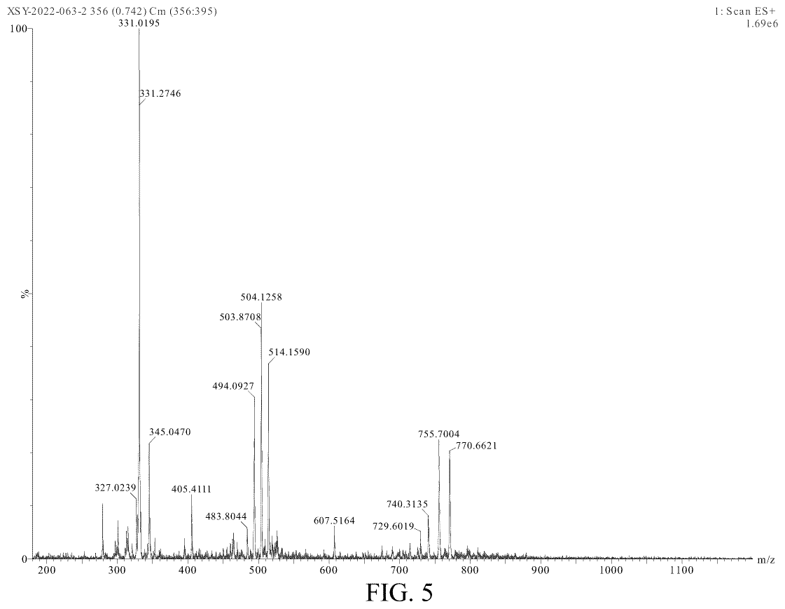

Polypeptide substituted with xylosyl at position 1 and glucosyl at positions 2 and 9:
the sequence characterization was as follows:
C(-Xyl)C(-Glu)RGDKGPDC(-Glu) (SEQ ID NO.8);
MS characterization was as follows: [M+2H]/2=755.7004 (see FIG. 5).

In summary, the present invention provided a method for glycosylation modification of proteins and/or polypeptides, and belonged to the field of medicinal chemistry. Using glycosyl sulfinates as raw materials, the present invention provided a method for glycosylation modification of proteins and/or polypeptides, which had some advantages, such as readily available reaction materials, mild reaction conditions, short reaction time, controllable reaction process, and high yield and purity of glycosylation-modified proteins and/or polypeptides. The glycosylation modification method of the present invention was not only applicable to proteins and/or polypeptides containing disulfide bonds, but also to proteins and/or polypeptides containing sulfhydryls, and even to proteins and/or polypeptides containing both disulfide bonds and sulfhydryls, with broad application prospects.

1. A method for glycosylation modification of proteins and/or polypeptides, characterized in that the method comprises the following steps:
the starting materials including glycosyl sulfinates, proteins and/or polypeptides, and oxidants are allowed to react in a solvent, to obtain glycosylation-modified proteins and/or polypeptides;
wherein, the proteins and/or polypeptides contain sulfhydryl and/or disulfide bonds;
the structure of the glycosyl sulfinate is as represented by formula W: 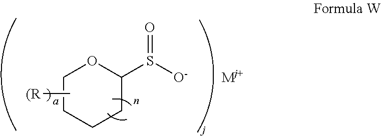
wherein, n is selected from 0 or 1;
a is selected from 3 or 4;
R is each independently selected from L₁Rₓ; or two adjacent R are linked to form a ring, while the other R is each independently selected from L₁Rₓ. The ring is either unsubstituted or substituted with one or more L₁Rₓ;
L₁ is selected from absence or C₁₋₂ alkylene, and Rₓ is selected from the group consisting of H, OH, C₁₋₆ alkyl, OAc (acetyloxy), OBn (benzyloxy), OR₈, NR₉R₁₀, 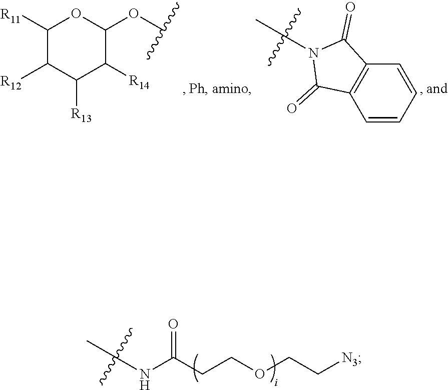
i is an integer selected from 0 to 6;
R₁₁, R₁₂, R₁₃, and R₁₄ are each independently selected from L₂R_{y}; L₂ is selected from absence or C₁₋₂ alkylene, and R_{y} is selected from the group consisting of H, OH, C₁₋₆ alkyl, OAc, OBn, OR₈, and NR₉R₁₀;
R₈ is selected from C₁₋₆ alkyl;
R₉ is selected from the group consisting of H, C₁₋₆ alkyl, Ac (acetyl), and Bn (benzyl);
R₁₀ is selected from the group consisting of H, C₁₋₆ alkyl, Ac, and Bn;
j is selected from 1, 2, or 3;
M^{j+} is a j-valent cation.

2. The method according to claim 1, characterized in that the oxidant is selected from one or more of hydrogen peroxide, tert-butyl hydroperoxide, potassium persulfate, oxygen, and tert-butyl peroxide;
and/or, the solvent is an aqueous solution, and preferably water or buffer;
and/or, the temperature of the reaction is room temperature;
and/or, the reaction is carried out under inert gas atmosphere.

3. The method according to claim 1, characterized in that the protein and/or polypeptide contains sulfhydryl, and the number of amino acid residues in the protein and/or polypeptide is 70-1000;
the method is method 1 or method 2;
method 1 comprises the following steps: first, the raw materials proteins and/or polypeptides containing sulfhydryl and compound A are allowed to react as the first step in a solvent, and then glycosyl sulfinates and oxidants are added for carrying out the second step reaction, to obtain glycosylation-modified proteins and/or peptides; compound A is 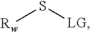
wherein LG is a leaving group, R_{w} is alkyl, aryl, or heteroaryl; alternatively, R_{w} and LG are linked to form a ring; preferably, compound A is 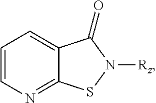
wherein R_{z} is C₁₋₈ alkyl, and preferably C₁₋₃ alkyl;
method 2 comprises the following steps: starting materials proteins and/or polypeptides containing sulfhydryl, glycosyl sulfinates, and oxidants are allowed to react in a solvent, to obtain glycosylation-modified proteins and/or polypeptides.

4. The method according to claim 3, characterized in that in method 1, the molar ratio of the protein and/or polypeptide containing sulfhydryl, compound A, glycosyl sulfinate, and oxidant is 1:(10-300):(20-600):(20-600), and preferably 1:200:400:400; and/or, the ratio of the protein and/or polypeptide containing sulfhydryl to the solvent is (0.05-0.5) μmol:1 mL, and preferably 0.1 μmol:1 mL; and/or, the time for the first step reaction is 1-20 minutes, and preferably 10 minutes; the time for the second step reaction is 0.2-1.5 hours, and preferably 1 hour;
in method 2, the molar ratio of the protein and/or polypeptide containing sulfhydryl, glycosyl sulfinate, and oxidant is 1:(20-600):(20-600), and preferably 1:400:400; and/or, the ratio of the protein and/or polypeptide containing sulfhydryl to the solvent is (0.05-0.5) μmol:1 mL, and preferably 0.1 μmol:1 mL; and/or, the reaction time is 0.2-1.5 hours, and preferably 1 hour.

5. The method according to claim 3, characterized in that the protein and/or polypeptide containing sulfhydryl is selected from the group consisting of sulfhydryl-containing Affibody, Mucin 1 protein, GTPase, sulfhydryl-containing non-structural protein, and sulfhydryl-containing amyloid protein;
preferably, the amino acid sequence of the sulfhydryl-containing Affibody is as set forth in SEQ ID NO.4.

6. The method according to claim 1, characterized in that the protein and/or polypeptide contains sulfhydryl, and the number of amino acid residues in the protein and/or polypeptide is 1-100;
the method is method 3 or method 4;
method 3 comprises the following steps: first, the raw materials proteins and/or polypeptides containing sulfhydryl and compound A are allowed to react in a solvent as the first step, and then glycosyl sulfinates and oxidants are added for carrying out the second step reaction to obtain glycosylation-modified proteins and/or polypeptides; compound A is 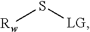
wherein LG is a leaving group, R_{w} is alkyl, aryl, or heteroaryl; alternatively, R_{w} and LG are linked to form a ring; preferably, compound A is 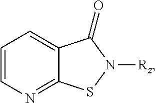
wherein R_{z} is C₁₋₈ alkyl, and preferably C₁₋₃ alkyl;
method 4 comprises the following steps: starting materials proteins and/or polypeptides containing sulfhydryl, glycosyl sulfinates, and oxidants are allowed to react in a solvent, to obtain glycosylation-modified proteins and/or polypeptides.

7. The method according to claim 6, characterized in that in method 3, the molar ratio of the protein and/or polypeptide containing sulfhydryl, compound A, glycosyl sulfinate, and oxidant is 1(1-3):(3-10):(3-10), and preferably 1:3:6:6; and/or, the ratio of the protein and/or polypeptide containing sulfhydryl to the solvent is (0.01-0.2) μmol:1 mL, and preferably 0.01 μmol:1 mL; and/or, the time for the first step reaction is 1-20 minutes, and preferably 10 minutes; the time for the second step reaction is 0.2-1.5 hours, and preferably 1 hour;
in method 4, the molar ratio of the protein and/or polypeptide containing sulfhydryl, glycosyl sulfinate, and oxidant is 1:(3-10):(3-10), and preferably 1:6:6; and/or, the ratio of the protein and/or polypeptide containing sulfhydryl to the solvent is (0.01-0.2) μmol:1 mL, and preferably 0.01 μmol:1 mL; and/or, the reaction time is 0.2-1.5 hours, and preferably 1 hour.

8. The method according to claim 6, characterized in that the protein and/or polypeptide containing sulfhydryl is selected from the group consisting of αVβ integrin-binding peptide, cell-penetrating peptide-R8, and reduced glutathione.

9. The method according to claim 1, characterized in that the protein and/or polypeptide contains disulfide bonds, and the number of amino acid residues in the protein and/or polypeptide is 2-2000;
the method comprises the following steps: glycosyl sulfinates, proteins and/or polypeptides containing disulfide bonds, and oxidants are allowed to react in a solvent, to obtain glycosylation-modified proteins and/or polypeptides.

10. The method according to claim 9, characterized in that the molar ratio of the protein and/or polypeptide containing disulfide bonds, glycosyl sulfinate, and oxidant is 1:(60-600):(20-600), and preferably 1:400:400;
and/or, the ratio of the protein and/or polypeptide containing disulfide bonds to the solvent is (0.05-0.5) μmol: 1 mL, and preferably 0.1 μmol: 1 mL;
and/or, the reaction time is 0.2-1.5 hours, and preferably 0.5-1 hour.

11. The method according to claim 9, characterized in that the protein and/or polypeptide containing disulfide bonds is selected from the group consisting of Herceptin, Inotuzumab ozogamicin, TGuard protein, Brentuximab vedotin, Mirvetuximab soravtansine, Upifitamab rilsodotin, Enfortumab vedotin, Certolizumab Gleevec, Telisotuzumab vedotin, Tusamitamab ravtansine, Ravtansine for treating thyroid adenomas, Recaticimab, amyloid P/A4 protein, Jag1 protein, lysozyme, iRGD peptide, and insulin.

12. The method according to claim 1, characterized in that the protein and/or polypeptide contains sulfhydryl and disulfide bonds;
the method is method 5 or method 6;
method 5 includes the following steps: first, proteins and/or polypeptides containing sulfhydryls and disulfide bonds, and compound A are used as raw materials for the first step and allowed to react in a solvent; and then glycosyl sulfinate a and oxidant are added to carry out the second step reaction, so as to obtain the proteins and/or polypeptides modified with glycosyl sulfinate a; and then glycosyl sulfinate b and oxidant are added to carry out the third step reaction, so as to obtain the proteins and/or polypeptides modified with glycosyl sulfinates a and b; compound A is 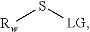
wherein LG is a leaving group, R_{w} is alkyl, aryl, or heteroaryl; alternatively, R_{w} and LG are linked to form a ring; preferably, compound A is 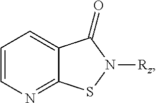
wherein R_{z} is C₁₋₈ alkyl, and preferably C₁₋₃ alkyl;
method 6 includes the following steps: first, using proteins and/or polypeptides containing sulfhydryls and disulfide bonds, glycosyl sulfinate a, and oxidants as raw materials, the first step reaction is carried out in a solvent to obtain proteins and/or polypeptides modified with glycosyl sulfinate a; then, glycosyl sulfinate b and oxidants are added to carry out the reaction in step (2), so as to obtain proteins and/or polypeptides modified with glycosyl sulfinates a and b;
glycosyl sulfinate a is the glycosyl sulfinate according to claim 1, while glycosyl sulfinate b is the glycosyl sulfinate according to claim 1, and glycosyl sulfinate a and glycosyl sulfinate b are the same or different.

13. The method according to claim 12, characterized in that: in method 5, the molar ratio of the protein and/or polypeptide containing sulfhydryls and disulfide bonds, compound A, glycosyl sulfinate a, glycosyl sulfinate b, the oxidant used in the second step reaction, and the oxidant used in the third step reaction is 1:(1-3):(1-3):(1-3):(1-3):(1-3), and preferably 1:1.2:1.2:2:1.6:2; and/or, the ratio of the protein and/or polypeptide containing sulfhydryls and disulfide bonds to the solvent is (0.001-0.01) mmol:1 mL, and preferably 0.005 mmol:1 mL; and/or, the time for the first step reaction is 5-20 minutes, and preferably 10 minutes; the time for the second step reaction is 0.2-1.5 hours, and preferably 1 hour; the time for the third step reaction is 0.2-1.5 hours, and preferably 1 hour;
in method 6, the molar ratio of the protein and/or polypeptide containing sulfhydryls and disulfide bonds, glycosyl sulfinate a, glycosyl sulfinate b, the oxidant used in the first step reaction, and the oxidant used in the second step reaction is 1:(1-3):(1-3):(1-3):(1-3), and preferably 1:1.2:2:1.6:2; and/or, the ratio of the protein and/or polypeptide containing sulfhydryls and disulfide bonds to the solvent is (0.001-0.01) mmol:1 mL, and preferably 0.005 mmol:1 mL; and/or, the time for the first step reaction is 0.2-1.5 hours, and preferably 1 hour; the time for the second step reaction is 0.2-1.5 hours, and preferably 1 hour;

14. The method according to claim 12, characterized in that the protein and/or polypeptide containing sulfhydryl and disulfide bonds is selected from the peptides having the amino acid sequence of CCRGDKGPDC, the peptides having the amino acid sequence of SKDACIRTCVMCDEQ, and Sublantin antimicrobial peptides.

15. The method according to claim 1, characterized in that the structure of the glycosyl sulfinate is as represented by formula I: 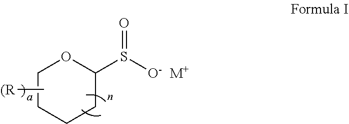
wherein, n is selected from 0 or 1;
a is selected from 3 or 4;
R is each independently selected from L₁Rₓ; or two adjacent R are linked to form a ring, while the other R is each independently selected from L₁Rₓ. The ring is either unsubstituted or substituted with one or more L₁Rₓ;
L₁ is selected from absence or C₁₋₂ alkylene, and Rₓ is selected from the group consisting of H, OH, C₁₋₆ alkyl, OAc, OBn, OR₈, NR₉R₁₀, 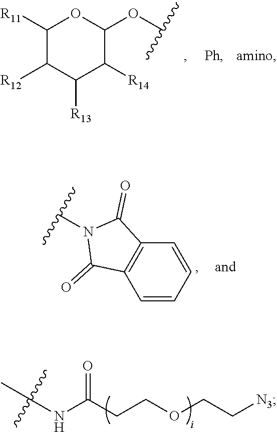
i is an integer selected from 0 to 6;
R₁₁, R₁₂, R₁₃, and R₁₄ are each independently selected from L₂R_{y}; L₂ is selected from absence or C₁₋₂ alkylene, and R_{y} is selected from the group consisting of H, OH, C₁₋₆ alkyl, OAc, OBn, OR₈, and NR₉R₁₀;
R₈ is selected from C₁₋₆ alkyl;
R₉ is selected from the group consisting of H, C₁₋₆ alkyl, Ac, and Bn;
R₁₀ is selected from the group consisting of H, C₁₋₆ alkyl, Ac, and Bn;
M⁺ is a monovalent cation.

16. The method according to claim 15, characterized in that the structure of the glycosyl sulfinate is as represented by formula II: 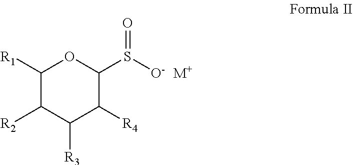
wherein, R₁, R₂, R₃, and R₄ are each independently selected from L₁Rₓ; alternatively, for R₁, R₂, R₃, and R₄, two adjacent groups are linked to form a ring, and the remaining two groups are each independently selected from L₁Rₓ. The ring is a 5-6-membered ring which is unsubstituted or substituted with one or more L₁Rₓ;
L₁ is selected from absence or methylene, and Rₓ is selected from the group consisting of H, OH, C₁₋₅ alkyl, OAc, OBn, OR₈, NR₉R₁₀, 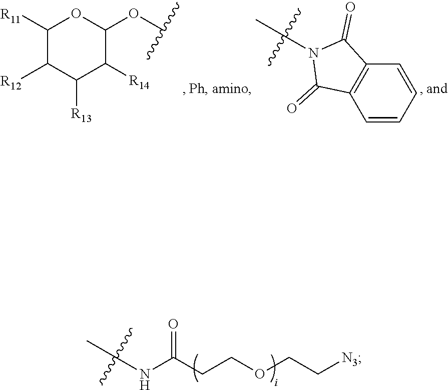
i is an integer selected from 0 to 4;
R₁₁, R₁₂, R₁₃, and R₁₄ are each independently selected from L₂R_{y}; L₂ is selected from absence or methylene, and R_{y} is selected from the group consisting of H, OH, C₁₋₅ alkyl, OAc, OBn, OR₈, and NR₉R₁₀;
R₈ is selected from C₁₋₅ alkyl;
R₉ is selected from the group consisting of H, C₁₋₅ alkyl, Ac, and Bn;
R₁₀ is selected from the group consisting of H, C₁₋₅ alkyl, Ac, and Bn;
M⁺ is a monovalent cation.

17. The method according to claim 15, characterized in that the structure of the glycosyl sulfinate is as represented by formula III: 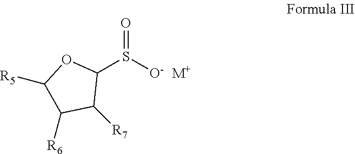
wherein, R₅, R₆, and R₇ are each independently selected from L₁Rₓ; alternatively, for R₅, R₆, and R₇, two adjacent groups are linked to form a ring, and the remaining group is L₁Rₓ. The ring is a 5-6-membered ring which is unsubstituted or substituted with one or more L₁Rₓ;
L₁ is selected from absence or methylene, and Rₓ is selected from the group consisting of H, OH, C₁₋₅ alkyl, OAc, OBn, OR₈, NR₉R₁₀, 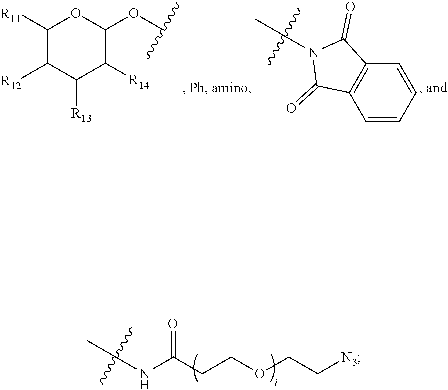
i is an integer selected from 0 to 4;
R₁₁, R₁₂, R₁₃, and R₁₄ are each independently selected from L₂R_{y}; L₂ is selected from absence or methylene, and R_{y} is selected from the group consisting of H, OH, C₁₋₅ alkyl, OAc, OBn, OR₈, and NR₉R₁₀;
R₈ is selected from C₁₋₅ alkyl;
R₉ is selected from the group consisting of H, C₁₋₅ alkyl, Ac, and Bn;
R₁₀ is selected from the group consisting of H, C₁₋₅ alkyl, Ac, and Bn;
M+ is a monovalent cation.

18. The method according to claim 16, characterized in that the 5-6-membered ring is a 5-6-membered saturated oxygen-containing heterocycle;
R₈ is selected from C₁₋₃ alkyl;
R₉ is selected from the group consisting of H, C₁₋₃ alkyl, Ac, and Bn;
R₁₀ is selected from the group consisting of H, C₁₋₃ alkyl, Ac, and Bn;
M⁺ is selected from the group consisting of Na⁺, K⁺, and Li⁺.

19. The method according to claim 1, characterized in that the structure of the glycosyl sulfinate is selected from the group consisting of:

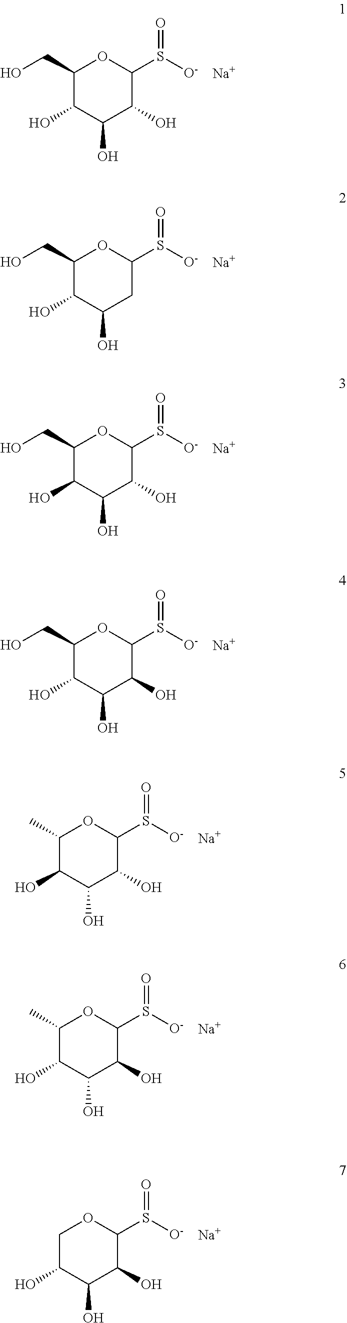

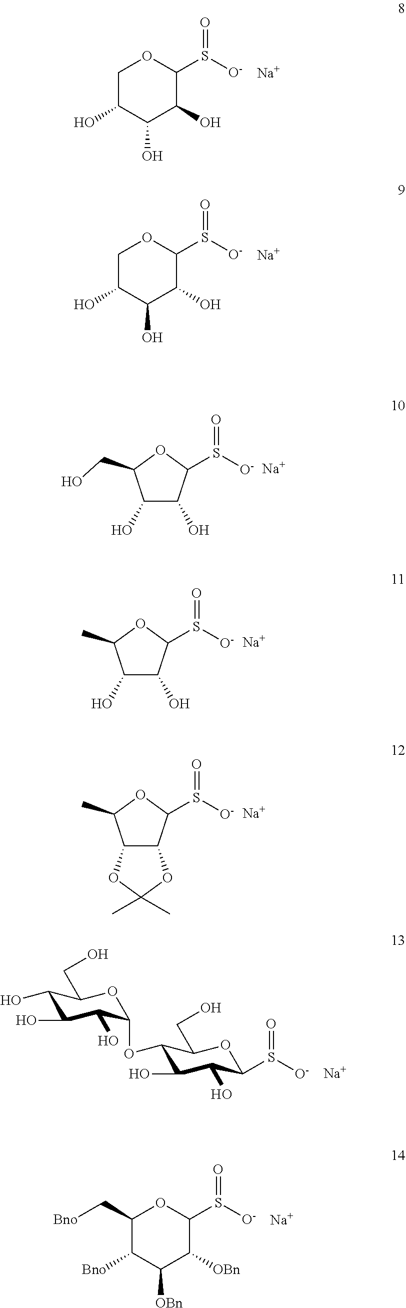

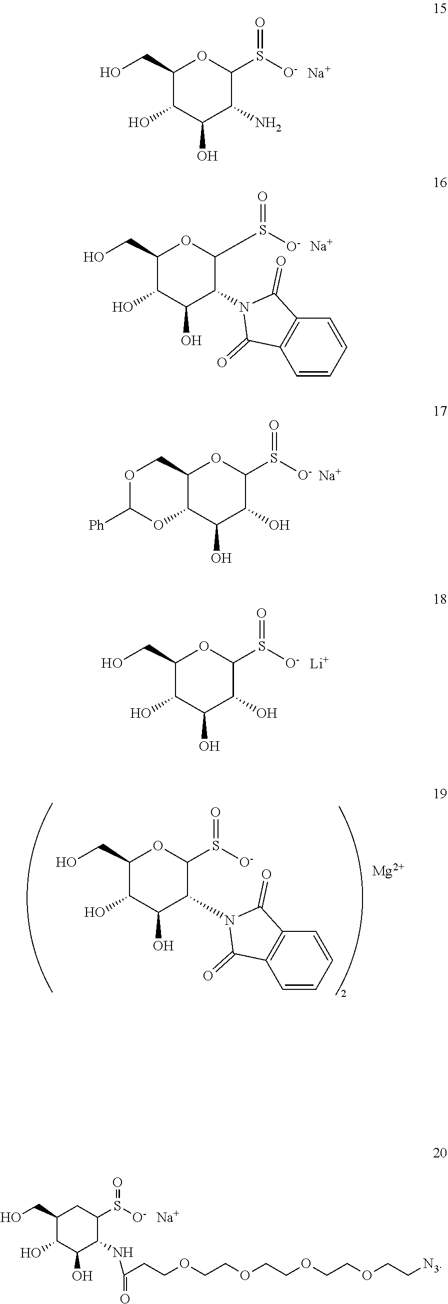

20. Glycosylation-modified proteins and/or polypeptides prepared by the method according to claim 1.
